(19) United States
(12) Patent Application Publication
(10) Pub. No.: US 2026/0144564 A1
(43) Pub. Date: May 28, 2026

(54) DEVICES AND METHODS FOR CUTTING A TENDON IN PROXIMITY TO A BICEPS

(71) Applicant: T.A.G. Medical Products Corporation Ltd., Kibbutz Gaaton (IL)

(72) Inventors: Uri BARAK, Nesher (IL); Moshe ESHKOL, Harutzim (IL); Hagay BOTANSKY, Haifa (IL); Avraham CHEZAR, Kibbutz Ramat HaShofet (IL); Noam Shlomo RESHEF, Tzurit (IL)

(73) Assignee: T.A.G. Medical Products Corporation Ltd., Kibbutz Gaaton (IL)

(21) Appl. No.: 19/120,804

(22) PCT Filed: Oct. 13, 2023
(86) PCT No.: PCT/IL2023/051073

§ 371 Date: Apr. 14, 2025

Related U.S. Application Data
(60) Provisional application No. 63/416,410, filed on Oct. 14, 2022.

(51) Int. Cl. A61B 17/32; A61B 17/00; A61B 90/00

(52) U.S. Cl. CPC A61B 17/32; A61B 90/03; A61B 2017/00367; A61B 2017/320064; A61B 2090/036

(57) ABSTRACT

A method for cutting an end portion of a long head of biceps (LHB) tendon from a biceps, comprising mounting a tendon cutting device having a cutter to the tendon, moving the tendon cutting device in a distal direction relative to a shoulder along the tendon towards the biceps, abutting the tendon cutting device to the biceps, thereby aligning a cutter location associated with a distal end of the tendon cutting device with a cutting location on the tendon adjacent the biceps, and cutting the tendon at the cutting location using the cutter.

### RELATED APPLICATION/S

This application claims the benefit of priority of U.S. Provisional Ser. No. 63/416,410 filed on 14 Oct. 2022, the contents of which are incorporated herein by reference in their entirety.

### FIELD AND BACKGROUND OF THE INVENTION

The present invention, in some embodiments thereof, relates to devices and methods of cutting a tendon and, more particularly, but not exclusively, to cutting a long head of biceps (LHB) tendon.

Surgical procedures for the cutting of a long head of biceps tendon are generally employed with the intent of using the severed tendon for treatment of a torn rotator cuff or similar ailments. In some cases, an entire tendon may be removed for application elsewhere. In other cases, the tendon is cut at one end and the partially attached tendon is applied to a point around the shoulder.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A method for cutting an end of a long head of biceps (LHB) tendon from a biceps, comprising:
mounting a tendon cutting device to a long head of biceps tendon;
moving the tendon cutting device in a distal direction along the tendon towards a biceps;
abutting the tendon cutting device to the biceps, thereby aligning a cutter location associated with a distal end of the tendon cutting device with a cutting location on the tendon adjacent the biceps; and,
cutting the tendon at the cutting location using the cutter.

Example 2. The method according to Example 1, further including securing the tendon after the mounting.

Example 3. The method according to either one of Examples 1 or 2, wherein the cutting is performed by relative rotation of the cutter located at a distal tip of the tendon cutting device.

Example 4. The method according to any one of Examples 1-3, wherein the cutting is performed by moving the cutter located near a distal tip of the tendon cutting device at a predetermined angle relative to the tendon.

Example 5. The method according to Example 2, wherein the securing is performed after pushing a first release handle associated with the tendon cutting device.

Example 6. The method according to Example 3, wherein the relative rotation is performed after pushing a second release handle associated with the tendon cutting device.

Example 7. The method according to Example 4, wherein the moving is performed by pushing a handle associated with the tendon cutting device.

Example 8. The method according to any one of Examples 1-Example 1, wherein the cutting is performed by moving the cutter located near a distal tip of the tendon cutting device at a perpendicular angle relative to the tendon.

Example 9. The method according to any one of Examples 1-8, wherein the cutter is realized as a mechanically deployable knife.

Example 10. A device for cutting an end of a long head of biceps (LHB) tendon, comprising:
an elongate body mountable on a tendon, including a tip at a distal end of the body, the tip shaped as a channel to receive a tendon and adapted to cease motion on the tendon when a distal end of the tip abuts a thickened portion of a biceps; and,
a cutter associated with the body and located at a predetermined distance from the distal end of the tip.

Example 11. The device according to Example 10, wherein the deployable cutter is realized as a cutting tool in the tip.

Example 12. The device according to either one of Examples 10 and 11, wherein the deployable cutter is located 1-5 mm before the distal end of the tip and is adapted to cut the tendon at an angle other than 90 degrees relative to the tendon.

Example 13. The device according to any one of Examples 10-12, further including a securing bar for holding the tendon close to the tip.

Example 14. The device according to any one of Examples 10-11 and 13, wherein the deployable cutter is located 1-5 mm before the distal end of the tip and is adapted to cut the tendon at a perpendicular angle relative to the tendon.

Example 15. A method for cutting an end of a long head of biceps (LHB) tendon from a biceps, comprising:
mounting a tendon cutting device to a long head of biceps tendon;
moving the tendon cutting device in a distal direction along the tendon towards a biceps;
holding the tendon in proximity to a biceps; and,
pulling the tendon in a direction away from the biceps until the tendon is separated from the biceps.

Example 16. The method according to Example 15, wherein the pulling occurs in a general vicinity of a shoulder.

Example 17. The method according to either one of Examples 15 or 16, further including separating the tendon from associated bone prior to the pulling.

Example 18. The method according to any one of Examples 15-17, further including separating at least one ligament associated with the tendon.

Example 19. The device according to any one of Examples 10-14, wherein the cutter is deployable.

Example 20. The device according to any one of Examples 10-14 and 19, wherein the predetermined distance is between 1 and 5 mm.

Example 21. A method for cutting a long head of biceps tendon, including:
mounting a biceps cutter on a long head of biceps tendon via an anterolateral shoulder arthroscopy portal;
closing an opening of a biceps cutter; slide the biceps cutter distally over the tendon towards biceps muscle;
stopping sliding the tendon cutter when it is abutted at a proximal biceps muscle; pull the biceps cutter backwards a touch, to be sure not to cut the musculotendinous junction of the muscle itself;
cutting the tendon at a cutting location using the cutter, wherein the cutting is by relative rotation, at an oblique angle or at a perpendicular angle; and,
withdrawing biceps cutter and a cut long head of biceps tendon from the anterolateral portal.

Example 22. A method for cutting a long head of biceps tendon, including:
mount a biceps cutter over a long head of biceps tendon via an anterolateral shoulder arthroscopy portal;
closing an opening of the biceps cutter slide the biceps cutter proximally towards a glenoid labrum, preferably under arthroscopic supervision;
stopping sliding when the biceps cutter abuts against a glenoid labrum;
completing cutting of the long head of biceps tendon in a flush or oblique manner, the cut being as close as possible to the labrum; and,
withdrawing biceps cutter and a cut long head of biceps tendon via the anterolateral portal.

Example 23. A method for cutting an end portion of a long head of biceps (LHB) tendon from a biceps, comprising:
a. mounting a tendon cutting device having a cutter to the tendon;
b. moving the tendon cutting device in a distal direction relative to a shoulder along the tendon towards the biceps;
c. abutting the tendon cutting device to the biceps, thereby aligning a cutter location associated with a distal end of the tendon cutting device with a cutting location on the tendon adjacent the biceps; and
d. cutting the tendon at the cutting location using the cutter.
Example 24. The method according to Example 23, further including securing the tendon within the tendon cutting device after the mounting.

Example 25. The method according to either one of Examples 23 or 24, wherein the mounting includes securing the tendon within the tendon cutting device after the mounting.

Example 26. The method according to any one of Examples 23-25, wherein the securing includes securing the tendon within a securing channel of the tendon cutting device.

Example 27. The method according to any one of Examples 23-26, wherein the cutting is performed by relative rotation of the cutter.

Example 28. The method according to any one of Examples 23-27, wherein the tendon cutting device includes inner and outer portions rotatable relative to each other, the inner portion located radially inward relative to the outer portion, and wherein the cutter is located on the inner portion.

Example 29. The method according to any one of Examples 23-27, wherein the tendon cutting device includes inner and outer portions rotatable relative to each other, the inner portion located radially inward relative to the outer portion, and wherein the cutter is located on the outer portion.

Example 30. The method according to any one of Examples 23-27, wherein the cutter is stationary within the tendon cutting device and the cutting is performed by rotation of a rotatable portion of the tendon cutting device relative to the cutter.

Example 31. The method according to Example 30, wherein the tendon cutting device includes inner and outer portions rotatable relative to each other, the inner portion located radially inward relative to the outer portion, and wherein the cutter is located on the inner portion.

Example 32. The method according to Example 30, wherein the tendon cutting device includes inner and outer portions rotatable relative to each other, the inner portion located radially inward relative to the outer portion, and wherein the cutter is located on the outer portion.

Example 33. The method according to any one of Examples 23-32, wherein the cutting is performed by moving the cutter at a predetermined angle relative to the tendon.

Example 34. The method of Example 33, wherein the predetermined angle is 90 degrees.

Example 35. The method of Example 33, wherein the predetermined angle is other than 90 degrees.

Example 36. The method according to any one of Examples 23-26, wherein the cutter is defined on a stationary portion of the tendon cutting device and wherein the cutting is performed by moving a movable portion of the tendon cutting device axially toward the cutter, the movable portion forcing the tendon against the cutter.

Example 37. The method according to Example 36, wherein the stationary portion is positioned radially outward relative to the movable portion.

Example 38. The method according to Example 36, wherein the stationary portion is positioned radially inward relative to the movable portion.

Example 39. The method according to any one of Examples 24-38, wherein the securing is performed after or simultaneously with pushing a first release handle associated with the tendon cutting device.

Example 40. The method according to any one of Examples 27-35, wherein the relative rotation is performed after or simultaneously with pushing a second release handle associated with the tendon cutting device.

Example 41. The method according to any one of Examples 33-35, wherein the moving is performed by pushing a handle associated with the tendon cutting device.

Example 42. The method according to any one of Examples 23-26, wherein the cutter is a mechanically deployable knife.

Example 43. The method according to any one of Examples 23-42, wherein the mounting includes mounting the tendon cutting device on the tendon via an anterolateral shoulder arthroscopy portal;
wherein the moving includes sliding the tendon cutting device distally relative to the shoulder over the tendon;
wherein the sliding is stopped when the biceps cutter abuts the biceps;
wherein the cutting is by relative rotation, at an oblique angle or at a perpendicular angle relative to a tendon longitudinal axis; and
wherein the method includes withdrawing the tendon cutting device and a cut tendon from the anterolateral shoulder arthroscopy portal.

Example 44. The method according to Example 43, further including, after the stopping, moving the tendon cutting device proximally relative the shoulder a sufficient distance to ensure that a musculotendinous junction of the biceps muscle is not cut by the tendon cutting device.

Example 45. The method according to any one of Examples 23 and 42-43, further including, after the mounting, closing an opening of the tendon cutting device to secure the cutting location within the biceps cutter.

Example 46. A device for cutting an end portion of a long head of biceps (LHB) tendon from a biceps, comprising:
a. an elongate body mountable on the tendon, the elongate body including a tip and a cutter at a distal end of the body, the tip shaped as a channel to receive a portion of the tendon and adapted to move along the tendon and to cease the motion on the tendon when a distal end of the tip abuts a thickened portion of the biceps; and
b. a cutter location associated with the body and located at a predetermined distance from the distal end of the tip, the cutter configured to cut the tendon at the cutter location.

Example 47. The device according to Example 46, the device including a cutting tool in the tip.

Example 48. The device according to either one of Examples 46 and 47, wherein the cutter location is at a distance of from about 1 mm to about 5 mm from the distal end of the tip.

Example 49. The device according to any one of Examples 46-48, wherein the cutter is adapted to cut the tendon at a predetermined angle relative to the tendon.

Example 50. The device according to Example 49, wherein the predetermined angle is 90 degrees.

Example 51. The device according to Example 49, wherein the predetermined angle is other than 90 degrees.

Example 52. The device according to any one of Examples 46-51, wherein the device includes a stationary portion and a movable portion, wherein the cutter is defined on the stationary portion, and wherein the movable portion is configured to be moved toward the cutter and is configured to force the tendon against the cutter.

Example 53. The device according to Example 52, wherein the stationary portion is positioned radially outward relative to the movable portion.

Example 54. The device according to Example 52, wherein the stationary portion is positioned radially inward relative to the movable portion.

Example 55. The device according to any one of Examples 46-54, wherein the movable portion is configured to one of move axially and rotate.

Example 56. The device according to any one of Examples 46-51, wherein the device includes a stationary portion and a movable portion, wherein the cutter is defined on the movable portion, and wherein the movable portion is configured to be moved toward the stationary portion and is configured to force the tendon against the stationary portion.

Example 57. The device according to Example 56, wherein the stationary portion is positioned radially outward relative to the movable portion.

Example 58. The device according to Example 56, wherein the stationary portion is positioned radially inward relative to the movable portion.

Example 59. The device according to any one of Examples 56-58, wherein the movable portion is configured to one of move axially and rotate.

Example 60. The device according to any one of Examples 46-51, further including a securing bar for securing the tendon within the channel.

Example 61. The device according to any one of Examples 46-51, wherein the channel is sized and shaped to secure the tendon within the device.

Example 62. The device according to any one of Example 46-51 and 60-61, including a safety catch having a first orientation for preventing actuation of the device and a second orientation for allowing actuation of the device.

Example 63. The device according to Example 62, wherein the safety catch is configured to geometrically interlock with a portion of the device when in the first orientation.

Example 64. The device according to any one of Example 46-51 and 60-63, wherein the cutter is deployable.

Example 65. The device according to any one of Example 46-51 and 60-63, wherein the cutter is rotatable.

Example 66. The device according to any one of Examples 46-51 and 60-63, wherein the device includes a rotatable portion configured to force the tendon against the cutter.

Example 67. The device according to any one of Examples 46-66, wherein the predetermined distance is from about 1 mm to about 5 mm.

Example 68. A method for cutting or tearing an end of a long head of biceps (LHB) tendon from a biceps, comprising:
a. mounting a long head of biceps tendon on tendon cutting or tearing device;
b. moving the tendon cutting or tearing device in a distal direction relative to a shoulder along the tendon towards a biceps;
c. holding the tendon with the tendon cutting or tearing device in proximity to a biceps; and,
d. pulling the tendon in a direction away from the biceps, such that the tendon is separated from the biceps.

Example 69. The method of Example 68, wherein the holding is performed by a distal portion of the tendon cutting or tearing device, a blade on said tendon cutting or tearing device positioned adjacent the distal portion of the tendon cutting or tearing device.

Example 70. The method of either one of Examples 68 or 69, wherein the pulling includes pulling the tendon against a blade of the tendon cutting or tearing device.

Example 71. The method according to any one of Examples 68-70, wherein the pulling occurs in a general vicinity of a shoulder.

Example 72. The method according to any one of Examples 68-71, further including separating the tendon from an associated bone prior to the pulling.

Example 73. The method according to any one of Example 68-73, further including separating at least one ligament associated with the tendon from the shoulder.

Example 74. A method for cutting a long-head of biceps tendon, including:
a. mounting a biceps cutter over the tendon via an anterolateral shoulder arthroscopy portal;
b. sliding the biceps cutter towards a glenoid labrum;
c. stopping the sliding when the biceps cutter abuts against a glenoid labrum;
d. cutting the tendon at a cutting location on the tendon, in a flush or oblique manner, adjacent the glenoid labrum; and,
e. withdrawing biceps cutter and a cut end of the cut tendon via the anterolateral portal.

Example 75. The method according to Example 74, further including, after the sliding, closing an opening of the biceps cutter to secure the cutting location within the biceps cutter.

Example 76. The method according to either one of Examples 74 or 75, wherein the sliding is performed under arthroscopic supervision.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Some embodiments of the present invention may be described below with reference to flowchart illustrations and/or block diagrams of methods, apparatus (systems) and products according to embodiments of the invention. The instant invention, in some embodiments may be practiced with one or a plurality of elements or steps as described herein. It is understood that the instant invention may have application to other tendons, ligands, muscles, or other bodily parts and is herein described according to its application to a long head of biceps tendon solely for the purpose of clarity.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. Similar elements may have the same number, advanced by multiples of 100.

In the drawings:

FIG. 1 is a flowchart of an exemplary method for cutting a tendon using a tendon cutting device, according to some embodiments of the invention;

FIG. 2 is a flowchart of an exemplary method, according to some embodiments of the invention;

FIG. 3 is a perspective view of an exemplary long head of biceps tendon cutting device, according to some embodiments of the invention;

FIG. 4 is a perspective view of the tendon cutting device of FIG. 3, the device mounted on a long head of biceps tendon, according to some embodiments of the invention;

FIG. 5 is a perspective view of the tendon cutting device of FIG. 3, with a tendon secured thereto, according to some embodiments of the invention;

FIG. 6 is a perspective view of the tendon cutting device of FIG. 3, the device abutting a biceps, according to some embodiments of the invention;

FIG. 7 is a view of a portion of the tendon cutting device of FIG. 3, showing a cutter prior to cutting of the tendon, according to some embodiments of the invention;

FIG. 8 is a view of a portion of the tendon cutting device of FIG. 3, showing the cutter cutting the tendon, according to some embodiments of the invention;

FIG. 9 is a view of a portion of the tendon cutting device of FIG. 3, showing the tendon, after it has been cut by the tendon cutting device, according to some embodiments of the invention;

FIG. 10 is a view of a portion of an exemplary alternate embodiment of a tendon cutting device having a deployable cutter, according to some embodiments of the invention;

FIG. 11 is a perspective view of the tendon cutting device of FIG. 10, including an exemplary button for deploying the cutter, according to some embodiments of the invention;

FIG. 12 is a view of a portion of the tendon cutting device of FIG. 10, wherein the cutter has been fully deployed and a tendon has been cut, according to some embodiments of the invention;

FIG. 13 is a view of a portion of the tendon cutting device of FIG. 10, showing an exemplary mechanism for deployment of the cutter, according to some embodiments of the invention;

FIG. 14 is a view of a portion of the device of FIG. 10, showing a portion of an exemplary mechanism for deploying the cutter via an activation button, according to some embodiments of the invention;

FIG. 15 is a side view of an exemplary tendon cutting device, according to some embodiments of the invention;

FIG. 16 is a perspective view of a portion of the tendon cutting device of FIG. 15 having a channel in an open state, according to some embodiments of the invention;

FIG. 17 is a view of a portion of the tendon cutting device of FIG. 15 having a channel in a closed state, according to some embodiments of the invention;

FIG. 18 is a view of a portion of the device of FIG. 15, showing an exemplary activation pin of a cutter, according to some embodiments of the invention;

FIG. 19 is a view of a portion of the device of FIG. 15, showing an exemplary mechanism for deploying a cutter, according to some embodiments of the invention;

FIG. 20 is a view of a portion of the device of FIG. 15, showing the cutter after deployment thereof, according to some embodiments of the invention;

FIG. 21 is a side view of an exemplary a cutter tool, according to some embodiments of the invention;

FIG. 22 is a side view of an exemplary tendon grabber, according to some embodiments of the invention;

FIG. 23 is a flowchart of an alternative exemplary method for cutting a tendon using a tendon cutting device, according to some embodiments of the invention;

FIG. 24 is a flowchart of an alternative exemplary method for cutting a tendon using a tendon cutting device, according to some embodiments of the invention;

FIG. 25A is an exploded view of an exemplary tendon cutting device, according to some embodiments of the invention;

FIG. 25B is an enlargement of a portion of the tendon cutting device shown in FIG. 25A, according to some embodiments of the invention;

FIG. 25C is a side view of the device of the cutting device of FIG. 25A, shown in an initial operative orientation;

FIG. 25D is a side view of the device of the cutting device of FIG. 25A, shown in the initial operative orientation, wherein a tendon has been mounted thereon;

FIG. 25E is a side view of the tendon cutting device of FIG. 25A, shown after cutting a tendon, according to some embodiments of the invention;

FIG. 25F is an enlarged perspective view of a portion of the device of FIG. 25E, according to some embodiments of the invention;

FIG. 26A is a perspective view of an exemplary tendon cutting device, according to some embodiments of the invention;

FIG. 26B is an enlargement of a portion of the tendon cutting device shown in FIG. 26A, according to some embodiments of the invention;

FIG. 26C is a side view of the tendon cutting device of FIG. 26A, shown in an initial operative orientation, with a tendon having been mounted therein;

FIG. 26D is a side view of the tendon cutting device of FIG. 26A, shown after cutting a tendon, according to some embodiments of the invention;

FIG. 26E is an enlargement of a portion of the device of FIG. 26D, according to some embodiments of the invention;

FIG. 27A is a side view of an exemplary tendon cutting device having an actuation lever, according to some embodiments of the invention;

FIG. 27B is an exploded view of the tendon cutting device of FIG. 27A, according to some embodiments of the invention;

FIG. 27C is a side view of the tendon cutting device of FIG. 27A, having a tendon mounted thereon, according to some embodiments of the invention;

FIG. 27D is a side view of the tendon cutting device of FIG. 27C, after cutting of the tendon, according to some embodiments of the invention;

FIG. 28A is an exploded view of an exemplary tendon cutting device having an actuation button, according to some embodiments of the invention;

FIG. 28B is a side view of the tendon cutting device of FIG. 28A, according to some embodiments of the invention;

FIG. 28C is a side view of the tendon cutting device of FIG. 27B, in an initial operative orientation, the device having a tendon mounted thereon, according to some embodiments of the invention;

FIG. 28D is a side view of the tendon cutting device of FIG. 28C, after release of a safety catch, according to some embodiments of the invention;

FIG. 28E is a side view of the tendon cutting device of FIG. 28C, after cutting of the tendon, according to some embodiments of the invention;

FIG. 28F is an enlarged view of a portion of the tendon cutting device of FIG. 28E, according to some embodiments of the invention;

FIG. 29A is an exploded view of a tendon cutting device, according to some embodiments of the invention;

FIG. 29B is a side view of the tendon cutting device of FIG. 29A, according to some embodiments of the invention;

FIG. 29C is a side view of the tendon cutting device of FIG. 29B, having a tendon mounted thereon, according to some embodiments of the invention;

FIG. 29D is a side view of the tendon cutting device of FIG. 29C, after release of the safety catch, according to some embodiments of the invention; and

FIG. 29E is a side view of the tendon cutting device of FIG. 29C, after cutting of the tendon, according to some embodiments of the invention.

### DESCRIPTION OF SPECIFIC EMBODIMENTS OF THE INVENTION

The present invention, in some embodiments thereof, relates to cutting a tendon and, more particularly, but not exclusively, to methods and devices for cutting a long head of biceps tendon.

### Overview

An aspect of some embodiments of the invention relates to devices and methods for cutting a long head of biceps tendon in proximity to a biceps. Biological terms such as shoulder, tendon, ligament, biceps, and long head of biceps tendon may have their generally understood meanings in the medical arts. In some embodiments, the device allows for the precise cutting of a long head of biceps tendon in close proximity to a biceps to allow for a maximal length of severed tendon for use in a medical procedure. In some embodiments, the device rides along a long head of biceps tendon until a distal end of the device abuts a biceps. In some embodiments, abutting of biceps allows for cutting of the tendon within millimeters of the beginning of the biceps muscle. In some embodiments, as such, the resultant cut tendon is as long as is possible for maximal contribution to rotator cuff or other treatment for which such a partially disconnected tendon may generally be used. In some embodiments, additionally or alternatively, a user may cut a second terminus of the tendon at the shoulder end. Alternatively, the tendon cutting device and method may be used to cut a long head of biceps tendon first near the glenoid labrum and, optionally thereafter, to cut the tendon near the biceps, according to some embodiments, or vice versa.

An aspect of some embodiments of the invention relates to methods and devices for cutting a long head of biceps tendon immediately adjacent to a biceps. In some embodiments, the method for cutting of a long head of biceps (LHB) tendon from a biceps comprises either via an anterolateral shoulder arthroscopy portal or mini open approach, mounting a tendon cutting device on the long head of biceps tendon; optionally securing a tendon cutting location on the tendon, within the tendon cutting device; sliding the tendon cutting device distally, relative to a shoulder, along the tendon towards the biceps muscle; stopping sliding the tendon cutting device when it abuts the biceps muscle, thereby aligning a cutter location associated with a distal end of the tendon cutting device with the tendon cutting location adjacent the biceps; and cutting the tendon at the tendon cutting location using the cutter.

In some embodiments, the method, either via the anterolateral shoulder arthroscopy portal or mini open approach, comprises mounting the tendon cutting device on the long head of biceps tendon; optionally securing a cutting location on the tendon within the tendon cutting device; sliding the tendon cutting device proximally, relative to the shoulder, along the tendon towards the glenoid labrum; stopping sliding of the tendon cutting device when it abuts the glenoid labrum, thereby aligning a cutter location associated with a proximal end of the tendon cutting device with the tendon cutting location adjacent the glenoid labrum; and cutting the tendon at the tendon cutting location using the cutter.

An aspect of some embodiments of the invention relates to s method including securing the tendon within the tendon cutting device after the mounting. In some embodiments, the cutting is performed by rotation of a cutter located near a distal tip of the tendon cutting device relative to a stationary portion of the device, or by relative rotation of a portion of the device toward a stationary cutter. In some embodiments, the cutting is performed by deployment of a cutter located near a distal tip of the tendon cutting device at a predetermined angle relative to the tendon. In some embodiments, the device includes a stationary cutter, and a portion of the device is movable axially toward the cutter such that the tendon forced against the cutter by the axially movable portion. In some embodiments, the cutter is moved axially toward a stationary portion of the device and the cutter is forced against the tendon. In some embodiments, the cutter is realized as a mechanically deployable knife.

In some embodiments, optionally, securing is performed after pushing a first release handle associated with the tendon cutting device. In some embodiments, the relative rotation is performed after pushing a second release handle associated with the tendon cutting device. In some embodiments, the moving is performed by pushing a handle associated with the tendon cutting device.

In some embodiments, the cutting is performed by the cutter at a perpendicular angle relative to the tendon longitudinal axis. In some embodiments, the cutting is performed at an oblique angle relative to the tendon longitudinal axis. It will be appreciated by persons skilled in the art that it may be desirable to cut a tendon at a particular predetermined angle, depending on various factors such as, for example, the type of procedure being performed and how the cut end of the tendon is to be attached to another anatomical structure. In some embodiments, the cutting may be performed at a particular predetermined angle, thereby facilitating, for example, connection of the tendon to another anatomical structure.

An aspect of some embodiments of the invention relates to a device for cutting an end of a long head of biceps (LHB) tendon, comprising: an elongate body mountable on a tendon, including a tip at a distal end of the body, the elongate body having a portion shaped as a channel to receive a tendon, optionally adapted to secure the tendon within the device, and adapted to slide along the tendon. The body is adapted to cease motion on the tendon when a distal end of the tip abuts a thickened portion of a biceps; and a cutter associated with the body and located at a predetermined distance from the distal end of the tip.

In some embodiments, the cutter is realized as a cutting tool in the tip. In some embodiments, the predetermined distance is from about 1 mm to about 5 mm (optionally from about 0.5 mm to about 7 mm; optionally form about 0.1 mm to about 10mm) from the distal end of the tip and the cutter is adapted to cut the tendon at an angle other than 90 degrees relative to the tendon longitudinal axis. For example, the cutter may be adapted to cut the tendon at an angle in a range selected from about 1 degree to about 30 degrees, from about 30 degrees to about 50 degrees, from about 50 degrees to about 70 degrees, from about 70 degrees to about 89 degrees, from about 91 degrees to about 110 degrees, from about 110 degrees to about 130 degrees, from about 130 degrees to about 150 degrees, and from about 150 degrees to about 170 degrees. Alternatively, according to some embodiments, the cutter is adapted to cut the tendon at an angle of 90 degrees relative to the tendon longitudinal axis.

An aspect of some embodiments of the invention relates to a device including a securing bar for holding the tendon within the channel of the tendon cutting device. This may prevent the tendon from inadvertently being released from the channel, for example while sliding the device along the tendon toward the biceps, according to some embodiments. In some embodiments, the device comprises a securing bar for holding the tendon close to the tip, prior to cutting of the tendon.

An aspect of some embodiments of the invention relates to a device wherein a cutter is rotatable or a portion of the device is rotatable relative to the cutter. In some embodiments, the cutter is deployable. In some embodiments, the cutter is defined on a stationary portion of the device and a portion of the device is axially movable relative to the cutter. In some embodiments, the stationary portion is positioned radially outward relative to the movable portion. In some embodiments, the stationary portion is positioned radially inward relative to the movable portion.

In some embodiments, the cutter is located from about 1 mm to about 5 mm (optionally from about 0.5 mm to about 7 mm; optionally form about 0.1 mm to about 10mm) from the distal end of the tip and is adapted to cut the tendon at a perpendicular angle relative to the tendon longitudinal axis. In some embodiments, the cutter is adapted to cut the tendon at an oblique angle relative to the tendon longitudinal axis.

In some embodiments there is provided a safety catch to prevent inadvertent or premature actuation of the device, by preventing depression of an actuation lever or actuation button.

An aspect of some embodiments of the invention relates to a method for cutting an end of a long head of biceps (LHB) tendon from a biceps, comprising: mounting a tendon cutting device to a long head of biceps tendon; moving the tendon cutting device in a distal direction relative to a shoulder along the tendon towards a biceps; holding the tendon in proximity to a biceps; and, pulling the tendon in a direction away from the biceps, optionally against a blade of the tendon cutting device, until the tendon is separated from the biceps. In some embodiments, the method further comprises separating the tendon from associated bone prior to the pulling. In some embodiments, the method further comprises separating at least one ligament associated with the tendon.

An aspect of some embodiments of the invention relates to a method for cutting a long head of biceps tendon, including mounting a tendon cutting device on a long head of biceps tendon via an anterolateral shoulder arthroscopy portal; optionally closing an opening of the tendon cutting device to secure the tendon within the device; sliding the biceps cutter distally, relative to the shoulder, over the tendon towards the biceps muscle; stopping sliding of the tendon cutting device when the device abuts the biceps muscle; pulling the biceps cutter backwards slightly, for example, 1-2 mm, to potentially ensure that the musculotendinous junction of the muscle itself is not cut; cutting the tendon at a cutting location on the tendon using a cutter of the tendon cutting device, wherein the cutting is by relative rotation, at an oblique angle or at a perpendicular angle to the tendon longitudinal axis; and, withdrawing the tending cutting device and a cut long head of biceps tendon from the anterolateral portal.

An aspect of some embodiments of the invention relates to a method for cutting a long head of biceps tendon, including mounting a tendon cutting device over a long head of biceps tendon via an anterolateral shoulder arthroscopy portal; optionally closing an opening of the tendon cutting device; sliding the device proximally, relative to the shoulder, towards a glenoid labrum, optionally under arthroscopic supervision; stopping the sliding when the tendon cutting device abuts the glenoid labrum; cutting the long head of biceps tendon in a flush or oblique manner relative to the tendon longitudinal axis, optionally the cut being as close as possible to the labrum; and withdrawing tendon cutting device and a cut long head of biceps tendon via the anterolateral portal.

Before explaining at least one embodiment of the invention in detail, it is to be understood that the invention is not necessarily limited in its application to the details of construction and the arrangement of the components and/or methods set forth in the following description and/or illustrated in the Figures and/or the Examples. The invention is capable of other embodiments or of being practiced or carried out in various ways.

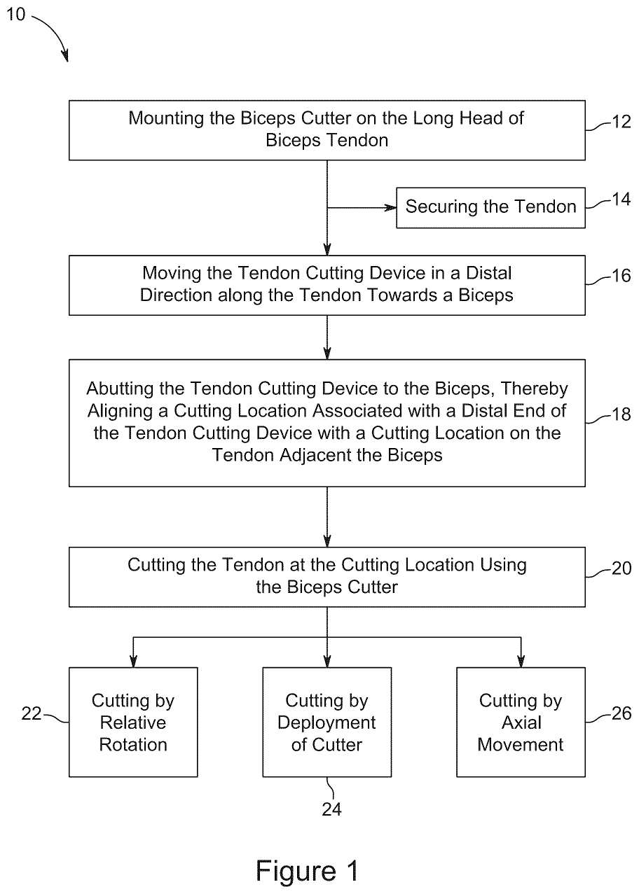

Referring now to the drawings, FIG. 1 is a flowchart of an exemplary method 10 for cutting a tendon using a tendon cutting device, according to some embodiments of the invention. In some embodiments, an exemplary method 10 for cutting an end of a long head of biceps (LHB) tendon from a biceps comprises: at 12, mounting a tendon cutting device to a long head of biceps tendon; at 16, moving the tendon cutting device in a distal direction, relative to a shoulder, along the tendon towards a biceps; at 18, abutting the tendon cutting device to the biceps, thereby aligning a cutter location associated with a distal end of the tendon cutting device with a cutting location on the tendon adjacent the biceps; and, at 20, cutting the tendon at the cutting location using the cutter. It has been found that abutting the tendon cutting device to the biceps allows cutting of the tendon to be performed at a location on the tendon as close as possible to the biceps muscle, thereby leaving at most a small portion of the tendon still attached to the biceps muscle. Alternatively, according to some embodiments, if it is desired to leave a portion of the tendon still attached to the biceps muscle, the device may be moved along the tendon toward the biceps muscle until it is a desired distance from the biceps muscle, and the tendon may be cut at that location, i.e., at the desired distance from the biceps muscle.

In some embodiments, the method optionally further comprises, at 14, securing the tendon within the tendon cutting device after the mounting. In some embodiments, the cutting is performed by rotation 22 of the cutter located at a distal tip of the tendon cutting device relative to a stationary portion of the device. Alternatively, in some embodiments, the cutting is performed by rotation of a portion of the device relative to a stationary cutter of the device. In some embodiments, the cutting is performed by deploying (24) a cutter located near a distal tip of the tendon cutting device at a predetermined angle relative to the tendon. In some embodiments, at 26, the cutting is performed by axial movement of an outer portion of the device relative to a stationary cutter (or axial movement of a cutter relative to a stationary outer portion), or by axial movement of an inner portion of the device relative to a stationary cutter (or axial movement of a cutter relative to a stationary inner portion).

In some embodiments, securing 14 is optionally performed after pushing a first release handle associated with the tendon cutting device. In some embodiments, the relative rotation 22 is performed after pushing a second release handle associated with the tendon cutting device. In some embodiments, the cutter deployment 24 or the axial movement 26 is performed by pushing a handle or button or other actuator associated with the tendon cutting device.

In some embodiments, the cutting is performed by the cutter at a perpendicular angle relative to the tendon longitudinal axis. In some embodiments, the cutting is performed by the cutter at an oblique angle to the tendon longitudinal axis. In some embodiments, the cutter is realized as a mechanically deployable knife.

In some embodiments, the method is performed by a tendon cutting device moving along a long head of biceps tendon in a direction away from a shoulder and towards a biceps. In some embodiments, the tendon cutting device is inserted into a body generally through a surgically-prepared incision in the general area of a shoulder. In some embodiments, optionally, the tendon cutting device can reversibly secure the tendon within the device by closing an element over the tendon, wherein the tendon sits over a groove or channel in the tendon cutting device. In some embodiments, optionally with the tendon thus secured, a user can move the tendon cutting device along the tendon until a distal end of the tendon cutting device abuts or otherwise bumps up against a widened portion of a biceps. In some embodiments, when the tendon cutting device reaches the biceps, an associated cutter is positioned to cut the tendon. In some embodiments, the spacing between the distal end of the tendon cutting device and the cutter is generally from about 2 mm to about 5 mm. Optionally, the spacing is from about 1 mm to about 7 mm. Optionally, the spacing is from about 0.5 mm to about 10 mm. In some embodiments, by placing the cutter close to the distal end of the cutting device, a maximum amount of tendon can be made available after cutting of the tendon proximate the biceps.

In some embodiments, the cutting of the tendon may be performed by a fixed or deployable cutter. In some embodiments, cutting may be made by a circular action of a cutter or knife near the distal end of the tendon cutting device. In some embodiments, additionally or alternatively, cutting may be performed by a reversibly deployable knife at an oblique or perpendicular angle relative to the tendon longitudinal axis.

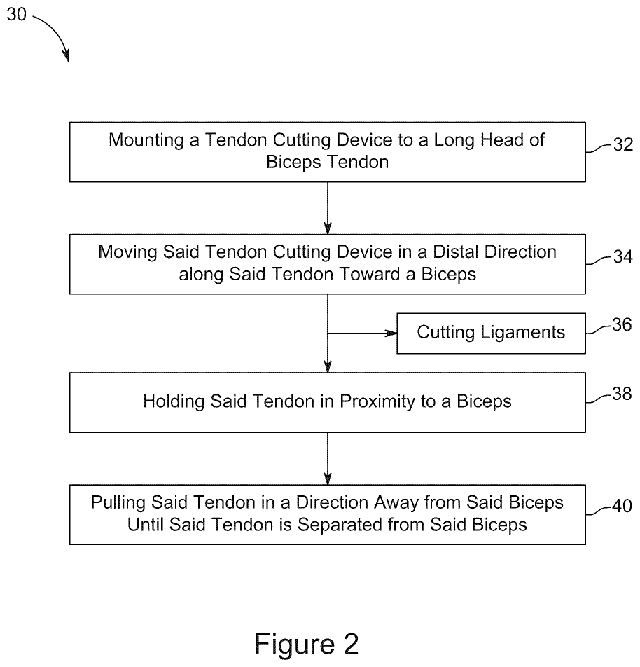

Referring now to FIG. 2, there is shown an exemplary method 30, according to some embodiments of the invention, for cutting an end of a long head of biceps (LHB) tendon adjacent a biceps, the method comprising, at 32, mounting a tendon cutting device to a long head of biceps tendon; at 34, moving the tendon cutting device in a distal direction, relative to a shoulder, along the tendon towards a biceps; at 38, holding the tendon in proximity to a biceps; and, at 40, pulling the tendon in a direction away from the biceps, optionally against a cutter of the device, until the tendon is separated from the biceps.

In some embodiments, the pulling occurs in a general vicinity of a shoulder. In some embodiments, the method further comprises separating the tendon from associated bone prior to the pulling. In some embodiments, the method further comprises, at 36, separating at least one ligament associated with the tendon.

In some embodiments, separation of a long head of biceps tendon from a biceps is generally not performed by cutting the tendon directly as explained above, according to some embodiments. In some embodiments, rather, ligaments associated with a long head of biceps may be either held or cut while the tendon is pulled from an opposite end in the general area of a shoulder and in a direction away from the biceps. In some embodiments, pulling of the tendon causes separation of the long head of biceps tendon from the biceps without a requirement to actively cut the tendon.

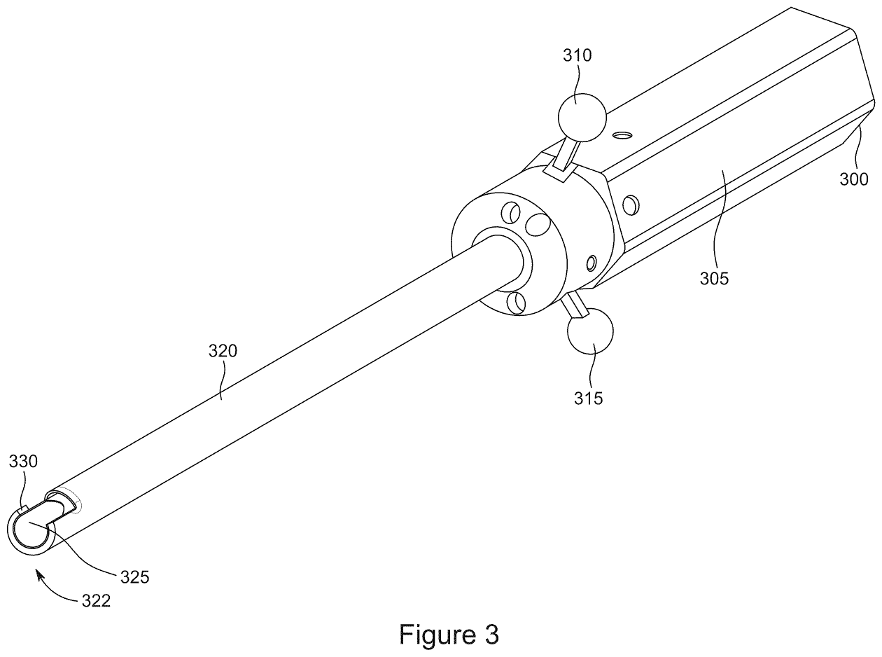

Referring now to FIG. 3, there is shown a perspective view of an exemplary tendon cutting device, according to some embodiments of the invention. In some embodiments, the tendon cutting device 300 includes a handle 305 for use by a physician/user. In some embodiments, the tendon cutting device 300 includes a first moveable button 310 that is used for optionally securing a tendon within the tendon cutting device 300 with a securing latch 545 (FIG. 5). In some embodiments, securing latch 545 may be deployed from device 500 to extend radially around tendon 535, thereby securing the tendon in channel 502, within device 300. According to some embodiments, a second moveable button 315 is adapted to actuate cutting of the tendon via relative rotational motion of a cutter (not shown). In some embodiments, the device 300 includes a handle 305 from which an elongate body 320 extends. In some embodiments, the elongate body has a distal end 322 that includes a channel 325 shaped to receive a tendon (not shown in this drawing). In some embodiments, at the distal end of the channel 325 is a distal tip 330 of the tendon cutting device 300. In some embodiments, the distal tip 330 is adapted to about a biceps (not shown in this drawing), as discussed further herein.

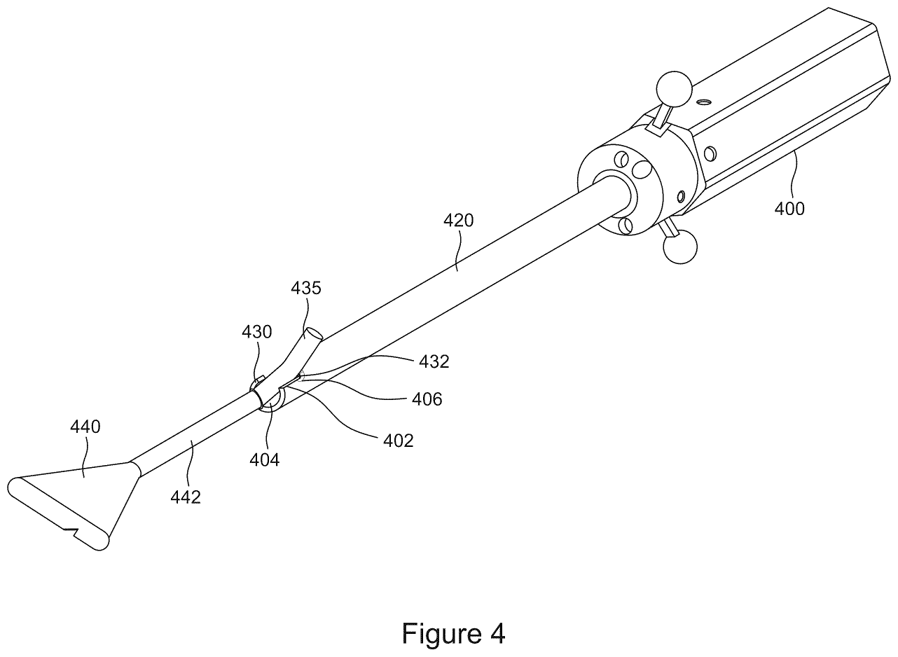

Referring now to FIG. 4, there is shown a perspective view of an exemplary tendon cutting device mounted on a long head of biceps tendon, according to some embodiments of the invention. Device 400 includes a channel 402 adapted for receiving a long head of biceps tendon 435 attached to a biceps 440. A distal end 404 of the channel 402 includes a first opening 430 into the device elongate body 420, according to some embodiments. A proximal end 406 of the channel includes a second opening 432, located at a lateral side of the device elongate body 420, according to some embodiments. According to some embodiments, the tendon 435 may be mounted in the device 400, in channel 402, such that it extends through the channel 402, from channel proximal end 406 to channel distal end 404. In some embodiments, a potential advantage of the channel 402 is that it is configured for receiving a tendon and for sliding the device 400 along the tendon as the cutting device 400 moves towards the biceps 440 prior to cutting, as discussed further herein.

It may be noted that, while the tendon 435 appears to be separated from the shoulder in some of the drawings, including FIG. 4, this is for simplicity only. It will be appreciated by persons skilled in the art that the tendon 435 may be attached to the shoulder as well as being connected to the biceps 440 during operation of the tendon cutting device, that the tendon 435 may be mounted on the tendon cutting device 400, and that the device 400 may be slid or otherwise moved along the tendon 435 while the tendon is attached at a first end 442 to the biceps and at a second end (not shown) to the shoulder, according to some embodiments.

Referring now to FIG. 5, there is shown a perspective view of an exemplary tendon cutting device 500 having a long head of biceps tendon 535 secured therein, according to some embodiments of the invention. The tendon cutting device 500 includes a securing latch 545 for securing a portion of the long head of biceps tendon 535. The securing latch 545 may be deployed via a button 510 in order to secure the tendon 535 within the tendon cutting device 500. In some embodiments, during a typical procedure for cutting a long head of biceps tendon, a physician will not have access to a camera or the like, so securing the tendon 535 to the tendon cutting device 500 via the securing latch 545 is an important feature for securing/retaining the tendon 535 within the tendon cutting device during a procedure. It may be noted that securing latch 545 allows the tendon 535 to be secured/retained within the channel 502 while allowing the device 500 to be slid along the tendon 535, for example, toward a biceps 540, according to some embodiments.

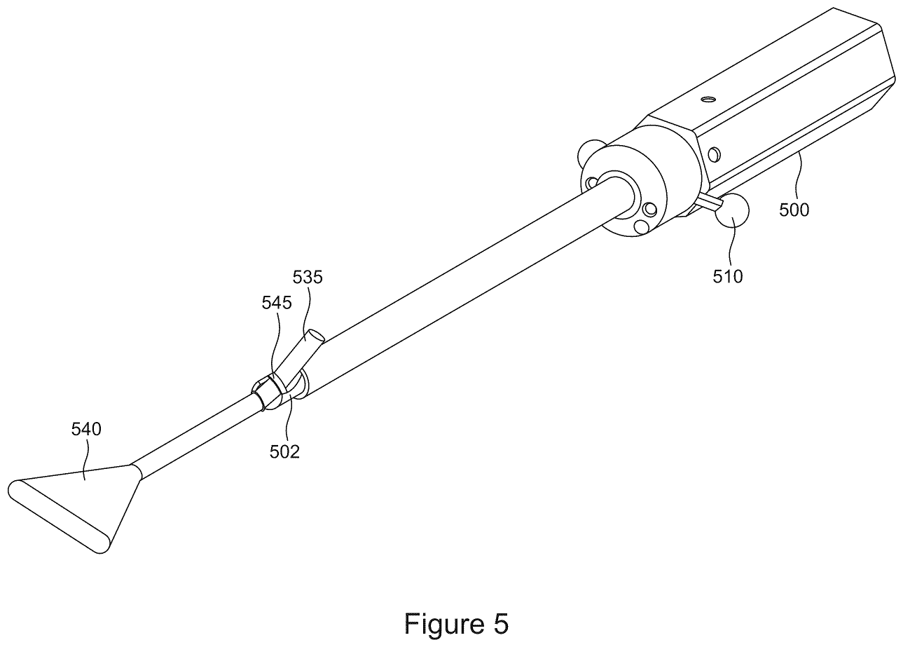

Referring now to FIG. 6, there is shown a perspective view of an exemplary tendon cutting device 600 shown abutting a biceps 640, according to some embodiments of the invention. FIG. 6 shows the tendon cutting device 600 and its associated secured tendon 635 mounted thereon, after the tendon cutting device 600 has been moved towards a biceps 640. In some embodiments, a distal end 630 of the tendon cutting device 600 abuts the widened muscle mass 642 of the biceps 640 and thus prevents further distal movement, relative to the shoulder (not shown), of the tendon cutting device 600 along the tendon 635. In some embodiments, when the tendon cutting device 600 can no longer move along the tendon 635 due to the widened portion 642 of the biceps 640, then the tendon cutting device 600 is in position to cut the tendon 635.

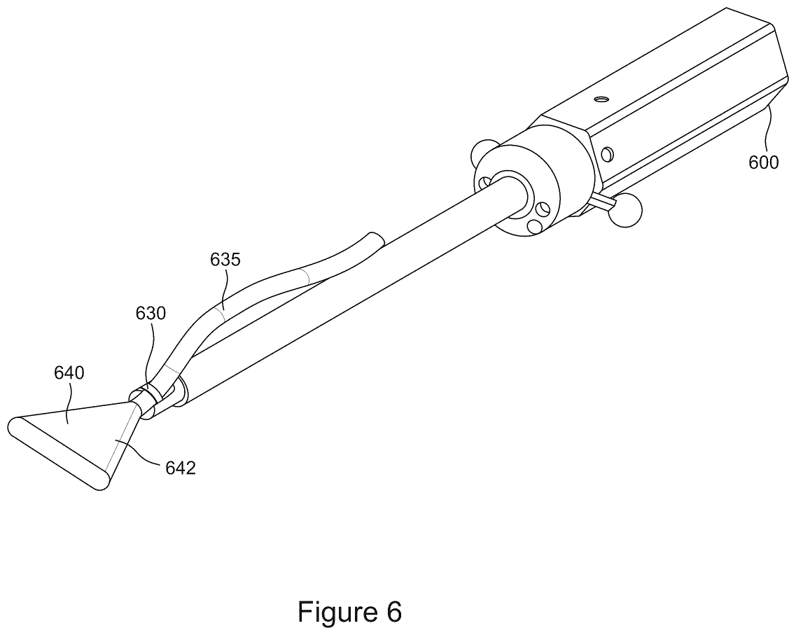

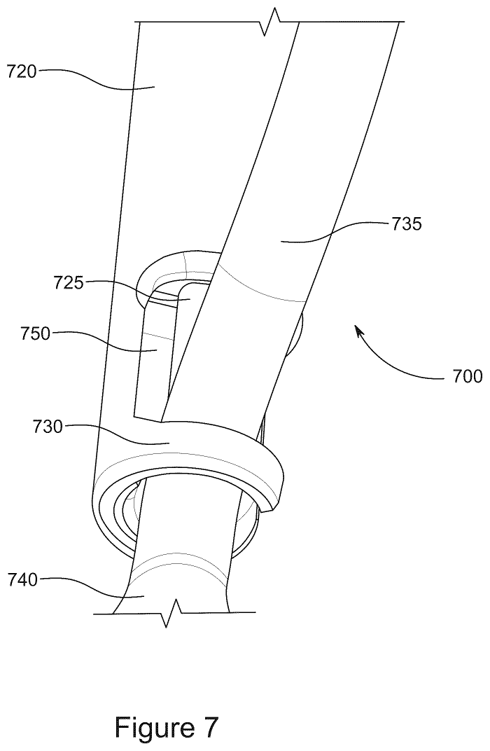

Referring now to FIG. 7, there is shown a portion of the tendon cutting device of FIG. 3, according to some embodiments of the invention. In some embodiments, the elongate body 720 (denoted by ref. no. 320 in FIG. 3) includes a cutter or cutting edge 750 proximal the distal end 730 of the tendon cutting device 700. In some embodiments, the cutter 750 is intentionally placed in a position relative to channel 725 so as not to cut the tendon 735 prior to positioning of the tendon proximate the biceps 740. This may allow the elongate body 720 to slide along the tendon 735 without damaging the tendon, according to some embodiments.

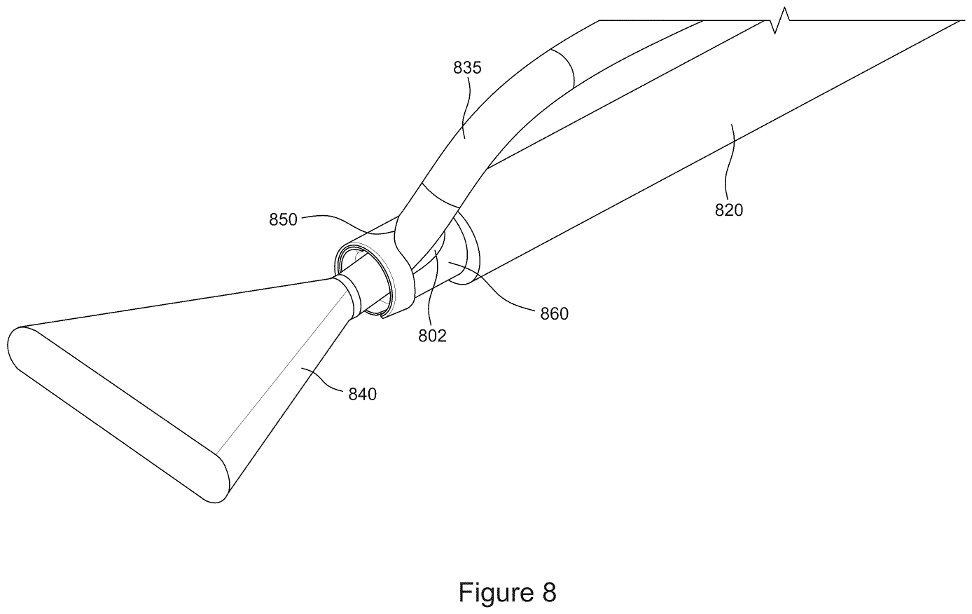

Referring now to FIG. 8, there is shown a portion of the tendon cutting device of FIG. 3, with a cutter 850 cutting a long head of biceps tendon adjacent a biceps 840, according to some embodiments of the invention. The device 800 includes an inner portion 860 which defines part of channel 802, whereby inner portion 860 is rotatable relative to elongate body 820, according to some embodiments. When tendon 835 is positioned within channel 802 and inner portion 860 is rotated relative to elongate body 820, it forces the tendon against cutter 850, thereby cutting through the tendon 835, according to some embodiments. It may be noted that the tendon may be cut at a distance of approximately about 2 mm to about 5 mm (optionally from about 1 mm to about 7 mm; optionally from about 0.5 mm to about 10mm) from the proximal (nearer the shoulder) end of the biceps 840. In some embodiments, cutting is actuated by a second moveable button 315 (FIG. 3), not visible in this view.

Alternatively, according to some embodiments, instead of the elongate body having a stationary cutting edge and the inner portion rotatable relative to the elongate body to force the tendon against the cutting edge, a cutting edge may be formed on a stationary inner portion, and an outer portion of the elongate body may be rotatable relative to an inner portion of the device to force the tendon against the cutting edge.

Further alternatively, according to some embodiments, instead of the elongate body having a stationary cutting edge and a rotatable portion adapted to force the tendon against the cutting edge, the device may include a stationary portion and a rotatable cutting edge adapted to cut the tendon against the stationary portion.

Referring now to FIG. 9, there is shown a portion of the tendon cutting device of FIG. 3, showing the tendon, after it has been cut by the tendon cutting device 900, according to some embodiments of the invention. FIG. 9 shows the tendon 935 fully separated from biceps 940 and no longer mounted within or secured to the tendon cutting device 900. In some embodiments, the cut tendon 935 is still attached at its second terminus and is shown unattached for ease of viewing only, as noted previously herein. In some embodiments, after cutting the tendon 935, the tendon cutting device 900 may be removed from the body (not shown).

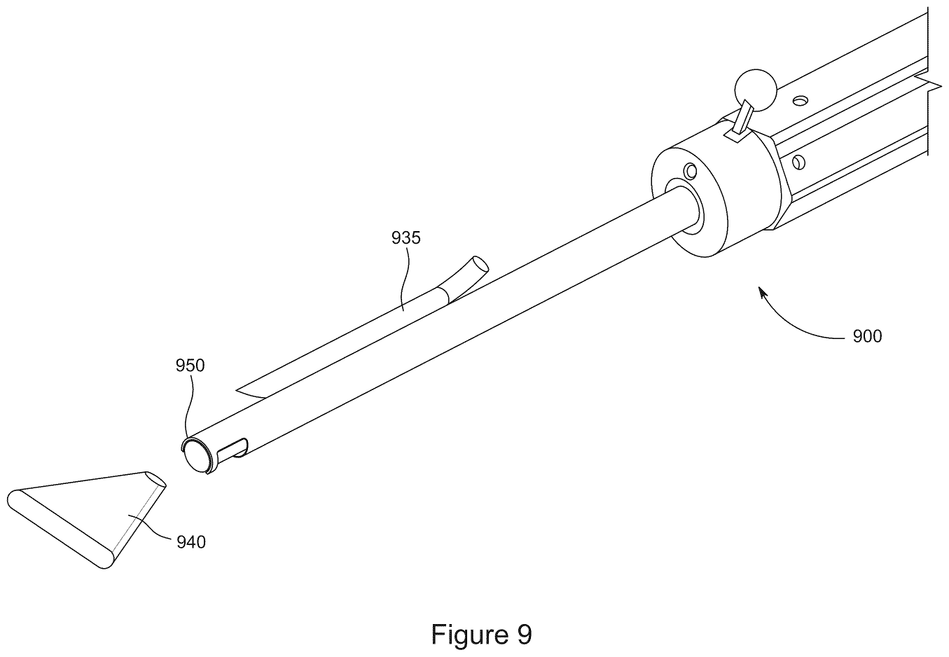

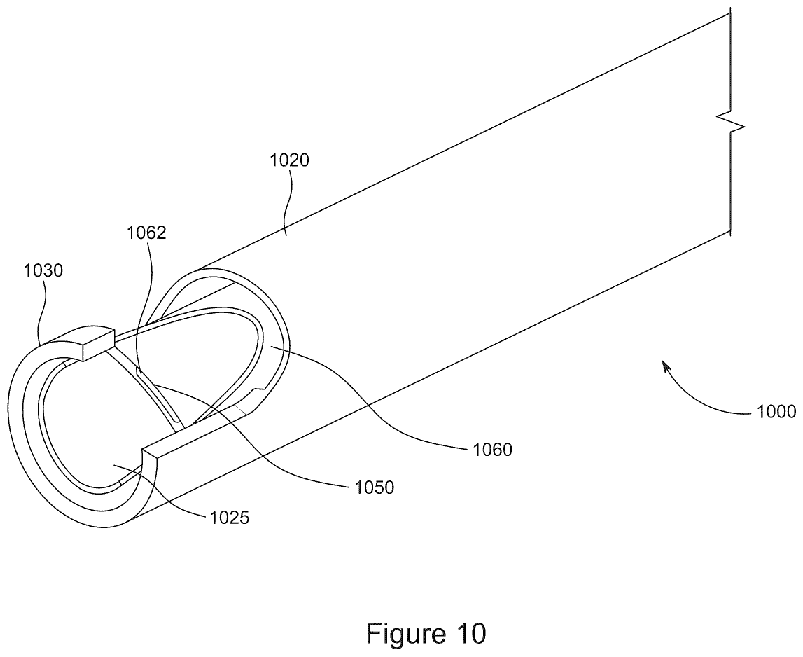

Referring now to FIG. 10, there is shown a portion of an exemplary alternate tendon cutting device 1000, according to some embodiments of the invention. According to some embodiments, the device includes an inner portion 1060 in which channel 1025 is formed. Alternatively, according to some embodiments, channel 1025 may be formed integrally as part of elongate body 1020. In some embodiments, a deployable cutter 1050 is positioned within the device 1000, outside the channel 1025, in order not to cut the tendon prematurely. According to some embodiments, device 1000 includes a slot 1062 through which cutter 1050 may be deployed at an angle relative to the elongate body 1020.

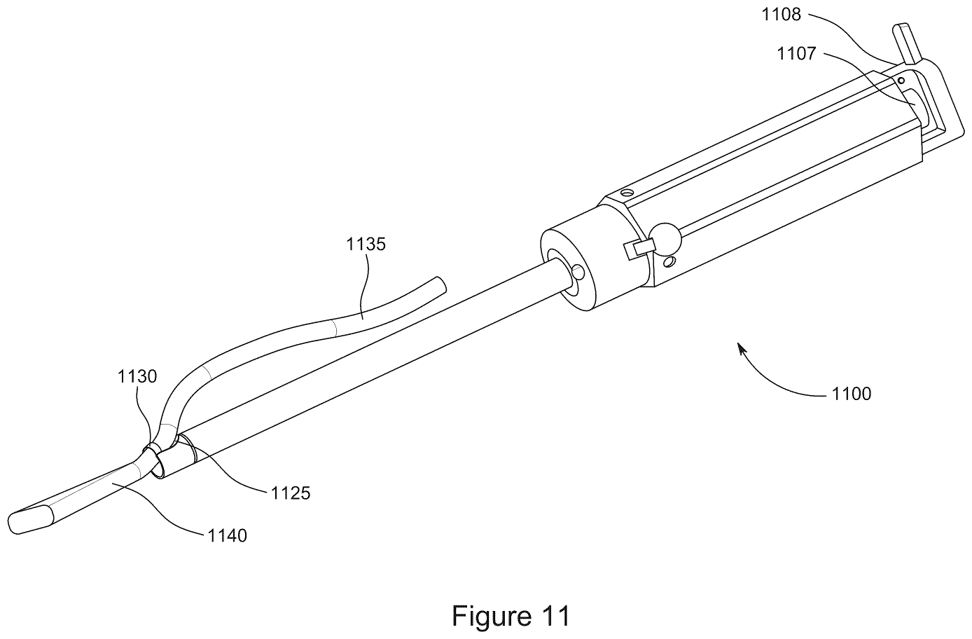

Referring now to FIG. 11, there is shown a perspective view of the device of FIG. 10, including an exemplary button 1107 for deploying the cutter (not shown in this drawing), according to some embodiments of the invention. FIG. 11 shows a tendon 1135 mounted on and optionally secured to a tendon cutting device 1100. In some embodiments, the tendon cutting device 1100 has at its proximal end a button 1107 adapted to deploy the cutter (not shown in this drawing) after the device distal tip 1130 contacts a biceps 1140, as discussed herein. In some embodiments, the button 1107 has an optional cover 1108 so as to prevent inadvertent or premature deployment of the cutter from within the channel 1125 holding the tendon 1135.

Optionally, any of the actuators discussed herein such as, for example, an actuator button, knob, or lever, may be provided with a cover to prevent inadvertent or premature actuation of the tendon cutting device, according to some embodiments.

Referring now to FIG. 12, there is shown a portion of the tendon cutting device of FIG. 10, wherein the cutter 1250 has been fully deployed, and the long head of biceps tendon 1235 has been severed from the biceps 1240, according to some embodiments of the invention. FIG. 12 shows a fully deployed cutter 1250 and corresponding fully depressed button 1207. In some embodiments, the tendon 1235 is separated from biceps 1240 and is no longer secured within the tendon cutting device 1200.

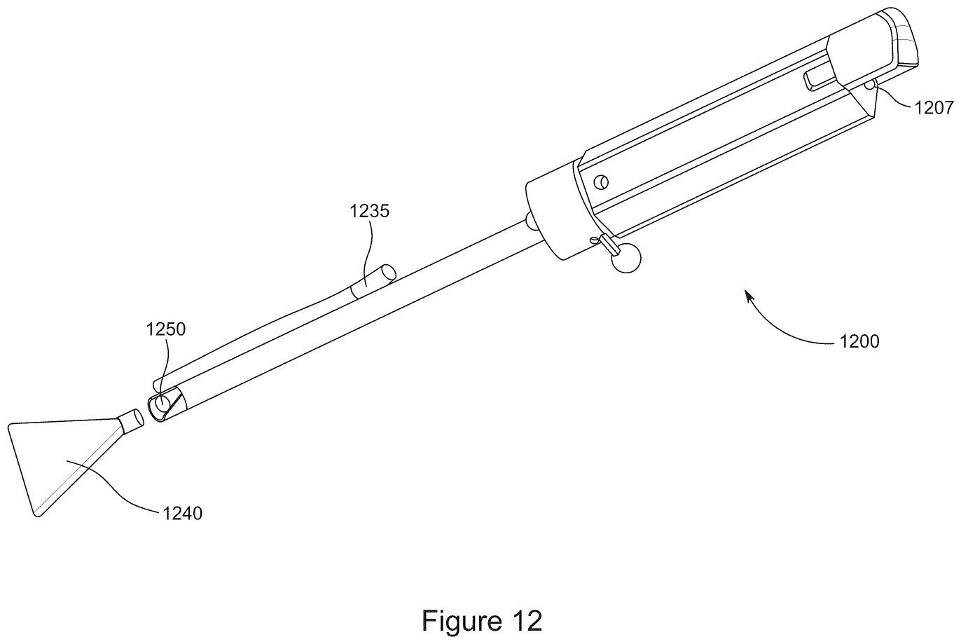

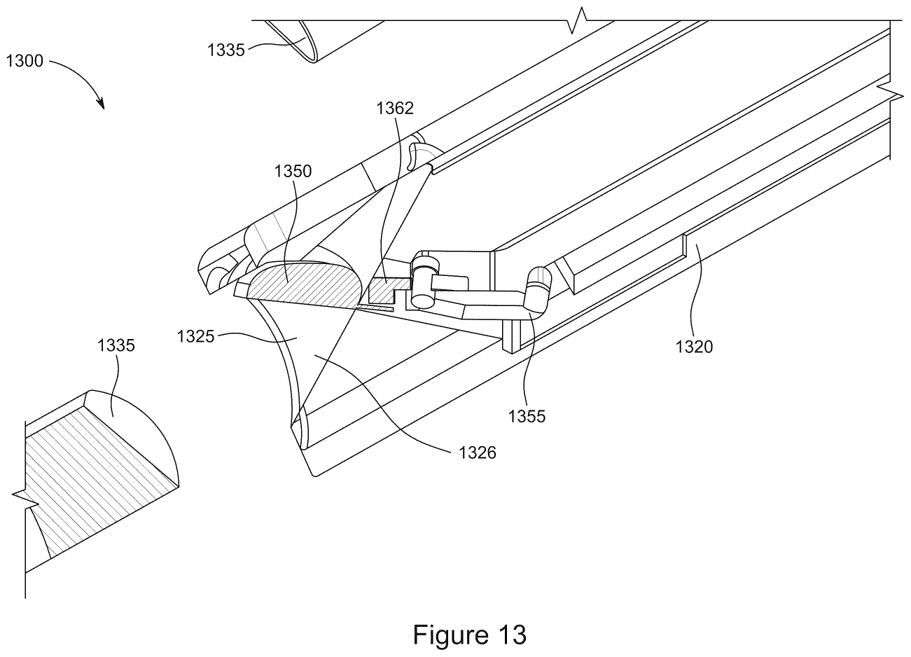

Referring now to FIG. 13, there is shown a portion of the tendon cutting device of FIG. 10, showing a portion of an exemplary mechanism 1355 for deployment of the cutter 1350 for cutting a tendon 1335, according to some embodiments of the invention. In some embodiments, the mechanism 1355 of device 1300 is activated by the button (1207 in FIG. 12) to push the cutter 1350 through slot 1362 in channel 1325 and move the cutter at an angle relative to the tendon 1335.

As seen in FIG. 13, due to the orientation of channel 1325, at a particular angle to the elongated body 1320 of the device 1300, when the tendon 1335 is mounted in channel 1325 it is oriented at the same angle relative to the elongated body 1320, such that the cutter 1350 is adapted to cut the tendon at an oblique angle to the tendon longitudinal axis, according to some embodiments. Optionally, if it is desired to cut the tendon 1335 at a different angle, the tendon may be moved such that it does not rest in a flush manner against the surface 1326 of the channel 1325, i.e., it may be held relative to the elongated body 1320 at an angle other than that defined by the surface 1326 of channel 1325, whereby deployment of the cutter 1350 will result in cutting of the tendon 1335 at a different angle such as, for example, a 90 degree angle relative to the tendon longitudinal axis, according to some embodiments. Similarly, regarding other embodiments of the invention discussed herein, the tendon may be cut at a 90 degree angle or at an oblique angle, depending on how it is held within the tendon cutting device, according to some embodiments.

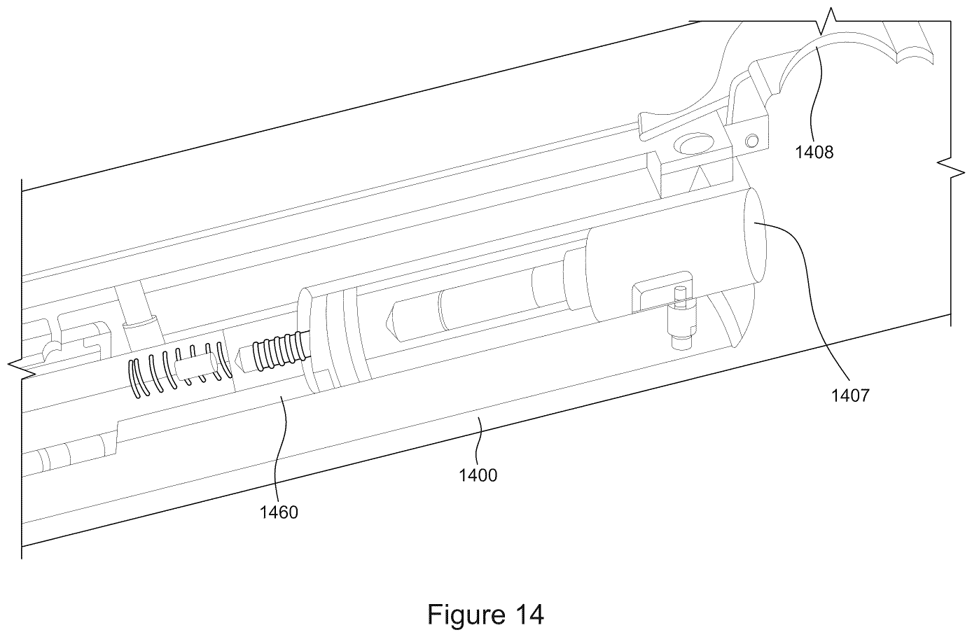

Referring now to FIG. 14, there is shown a portion of an exemplary mechanism for deploying the cutter via an activation button 1407, according to some embodiments of the invention. The button 1407 is shown in an initial position, and a cover 1408 (shown in FIG. 14 in an open position, and shown at 1108 in FIG. 11 in a closed position) is provided to prevent inadvertent or premature deployment of the cutter (1350 in FIG. 13) when the cover is in the closed position over button 1407, according to some embodiments. In some embodiments, depression of button 1407 of the tendon cutting device 1400 causes, through a series of springs, gears, and other mechanical features 1460, the cutter (See FIGS. 13, 1350) to deploy and cut tendon 1335 (FIG. 13). It is to be understood by persons skilled in the art that the mechanism shown and described with regard to FIGS. 13-14 is one example of a suitable mechanism for deploying the cutter, and that other mechanisms may be understood by persons skilled in the art to be similarly suitable, according to some embodiments.

It may be noted that this embodiment has one depressable button 1407 for optionally securing the tendon in the channel and for cutting the tendon, as distinguished from the previous embodiment (FIG. 3) that may have two sliding buttons as discussed herein.

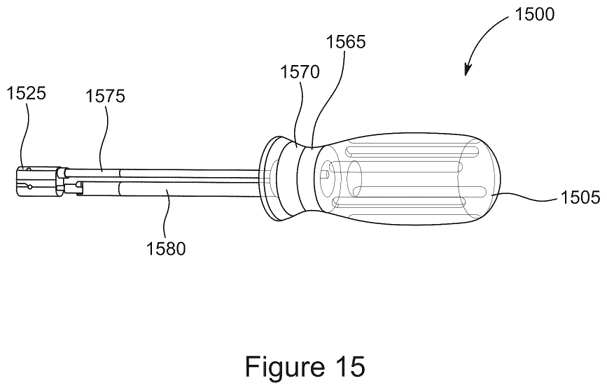

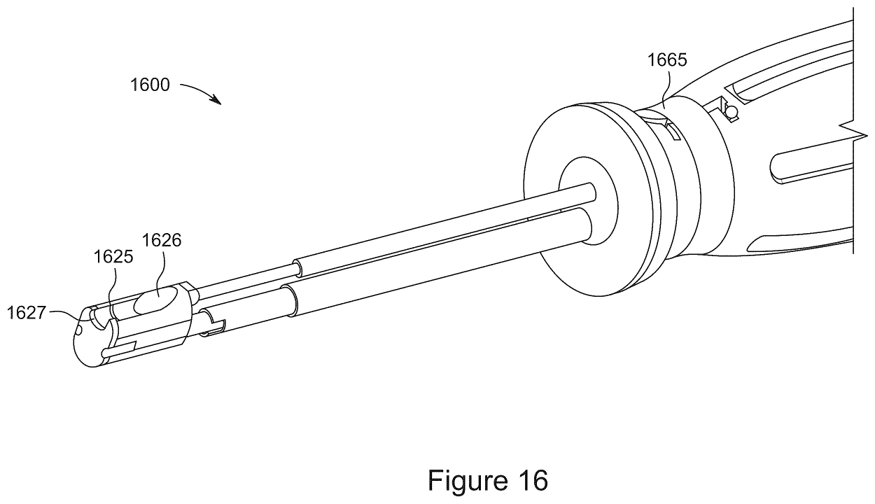

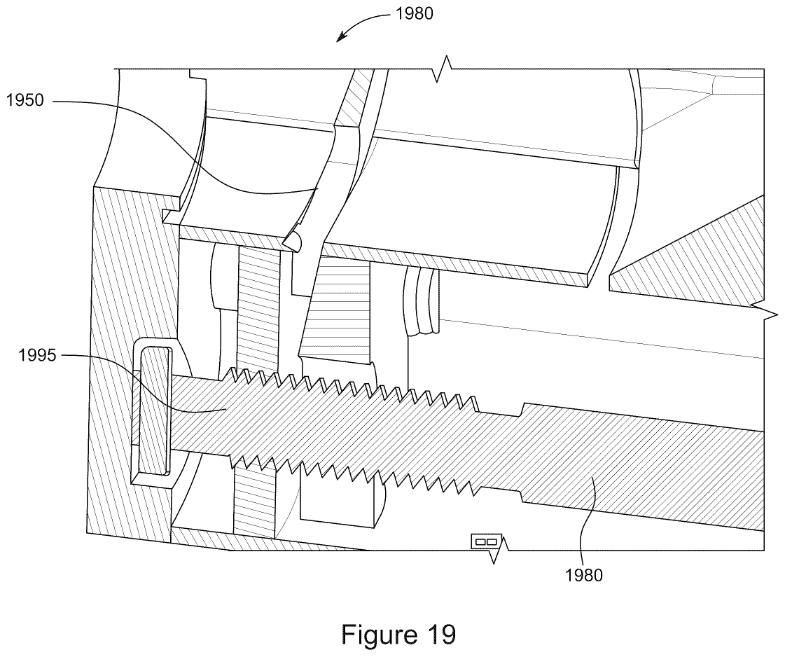

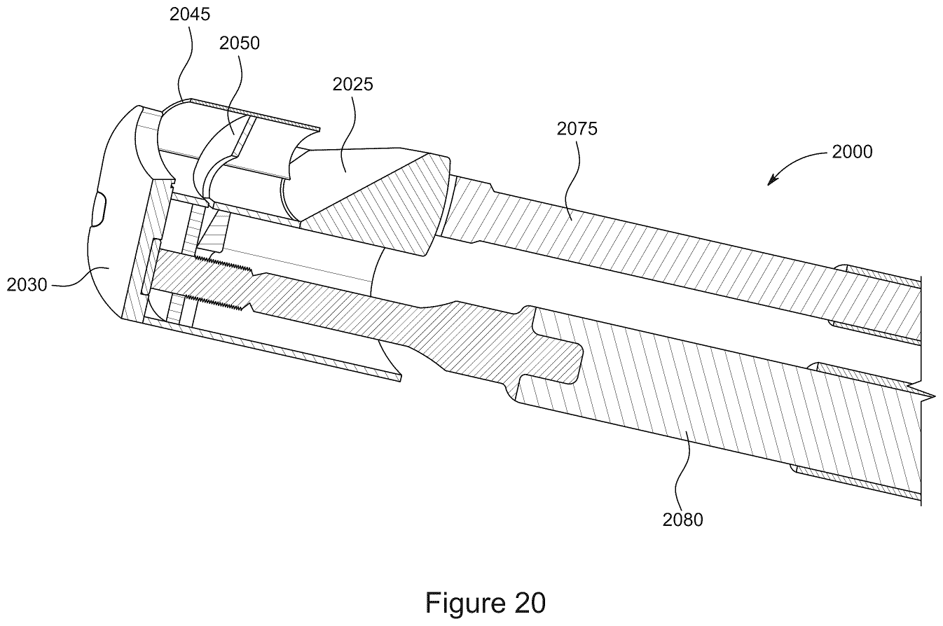

Referring now to FIG. 15, there is shown a side view of an exemplary tendon cutting device 1500, according to some embodiments of the invention. FIG. 15 shows an exemplary tendon cutting device 1500 including a handle 1505, a rotatable first ring 1565 and a second cutting ring 1570. In some embodiments, the cutting device 1500 further includes a first shaft 1575 that includes a channel 1525 adapted to receive a tendon, the channel including a proximal, angled surface 1626 and a distal surface 1627 (FIG. 16). and the device 1500 also includes a second shaft 1580 that includes a cutter (not shown). In some embodiments, this structure of device 1600 allows for a perpendicular cut of a tendon, as discuss further with regard to FIGS. 19-20.

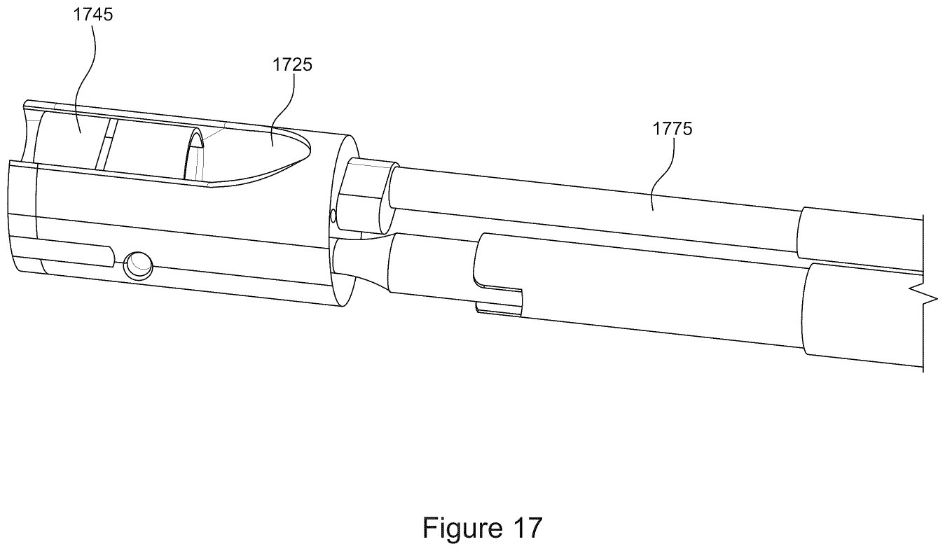

Referring now to FIG. 16, there is shown a perspective view of a portion of an exemplary tendon cutting device 1600 having a channel 1625 in an open state, according to some embodiments of the invention. In some embodiments, the channel 1625 includes an angled surface 1626 and a distal surface which is parallel to the device longitudinal axis. The channel 1625 is adapted to receive a tendon (not shown) therein. In some embodiments, a tendon may be optionally secured in the channel 1625 by a securing latch (1745 in FIG. 17) by rotation of the rotatable first ring 1665, for example, by one full rotation.

Referring now to FIG. 17, there is shown a view of a portion of an exemplary tendon cutting device having a channel 1725 in a closed state, according to some embodiments of the invention. FIG. 17 shows a securing latch 1745 in a closed position (without tendon), after rotation of shaft first rotatable ring 1665 (FIG. 16). In some embodiments, a full rotation of the first rotatable ring 1665 causes complete deployment of securing latch 1745. In some embodiments, the securing latch 1745 may hold a tendon in place in the channel 1725 as the tendon cutting device is moved towards biceps and/or while the tendon is being cut.

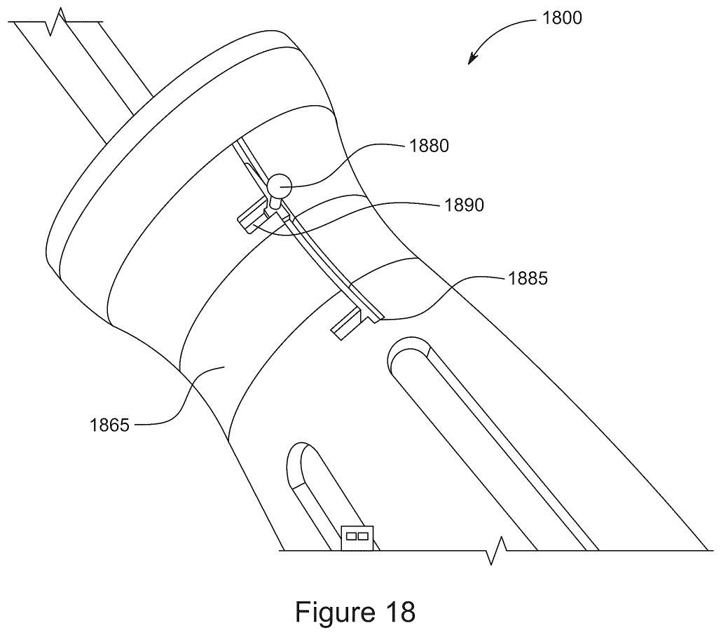

Referring now to FIG. 18, there is shown a portion of the tendon cutting device of FIG. 15, including an exemplary activation pin 1880 of a cutter (1950 in FIG. 19) in an exemplary tendon cutting device 1800, according to some embodiments of the invention. FIG. 18 shows pin 1880 and its first position 1885 prior to cutting of a tendon and in its second position 1890 after cutting of the tendon. In some embodiments, moving of pin 1880 from first position 1885 to second position 1890 causes deployment of a cutter (2050 in FIG. 20) adapted to cut a long head of biceps tendon at a ninety degree angle relative to the tendon longitudinal axis. In some embodiments, the pin 1880 can only move after rotation of the first rotatable ring 1865, to ensure that the securing latch 1745 has been closed over the tendon (not shown) in the channel 1725 (see FIG. 17), prior to deployment of the cutter.

Referring now to FIG. 19, there is shown a portion of the tendon cutting device of FIG. 15, including an exemplary mechanism for deploying a cutter 1950, according to some embodiments of the invention. FIG. 19 shows details of the second shaft 1980 wherein a gear system 1995 associated with the pin (see FIGS. 18, 1880) causes deployment of a cutter 1950 in a perpendicular orientation relative to tendon positioned in channel 1625 (FIG. 16). It is to be understood by persons skilled in the art that the mechanism shown and described with regard to FIG. 19 is one example of a suitable mechanism for deploying the cutter, and that other mechanisms may be understood by persons skilled in the art to be similarly suitable, according to some embodiments.

Referring now to FIG. 20, there is shown a portion of the device of FIG. 15, after deployment of an exemplary cutter 2050, according to some embodiments of the invention. In some embodiments, optionally, a closure or securing latch 2045 may be closed over a tendon, prior to deployment of the cutter 2050, thereby securing a tendon (not shown) in channel 2025, as discussed herein with regard to FIG. 17. For the sake of clarity, however, the securing latch 2045 is shown in an open position, so that the cutter 2050 is clearly shown. In some embodiments, a mechanism such as, for example, that contained in second shaft 2080 and discussed herein with regard to FIG. 19, leads to deployment of cutter 2050 for cutting a tendon at about 90 degrees to the tendon longitudinal axis. FIG. 20 shows the cutter 2050 fully deployed, but for the sake of clarity no tendon is shown. In some embodiments, opening and closing of the securing latch 2045 is controlled by the first rotatable ring (1665 in FIG. 16), as discussed herein, while deployment of the cutter 2050 is controlled by the moving of pin (1880 in FIG. 18) as previously described.

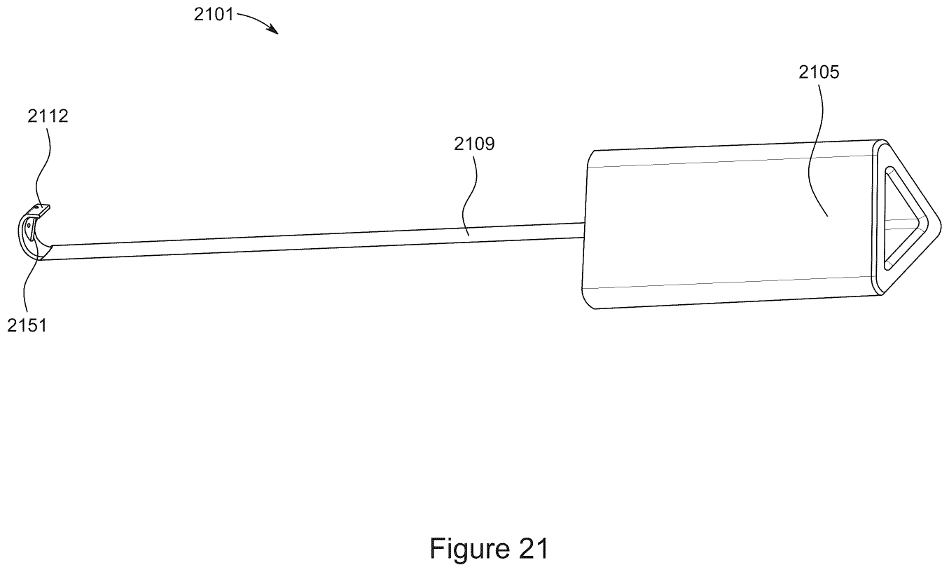

Referring now to FIG. 21, there is shown a side view of an exemplary a cutter tool 2102, according to some embodiments of the invention, that may be utilized for cutting or tearing off of a tendon such as, for example, a long head of biceps tendon. FIG. 21 shows a cutter tool 2101 including a handle 2105 and a metered shaft 2109 at the end of which is a semicircular end 2112 and optionally a blade 2151, for example, at a radially inner portion of the semicircular end 2112 of the cutter tool 2101. In some embodiments, the cutter tool 2101 may be generally inserted via an arthroscopic portal, the end 2112 with blade 2151 is placed under the tendon (with blade facing tendon), and the tool 2101 may be moved along the tendon until the device abuts a biceps and the blade 2151 is positioned adjacent a cutting location on the tendon. In order to cut or tear off the tendon, a transverse ligament is manually or mechanically pulled back, leading to separation of the tendon from the biceps.

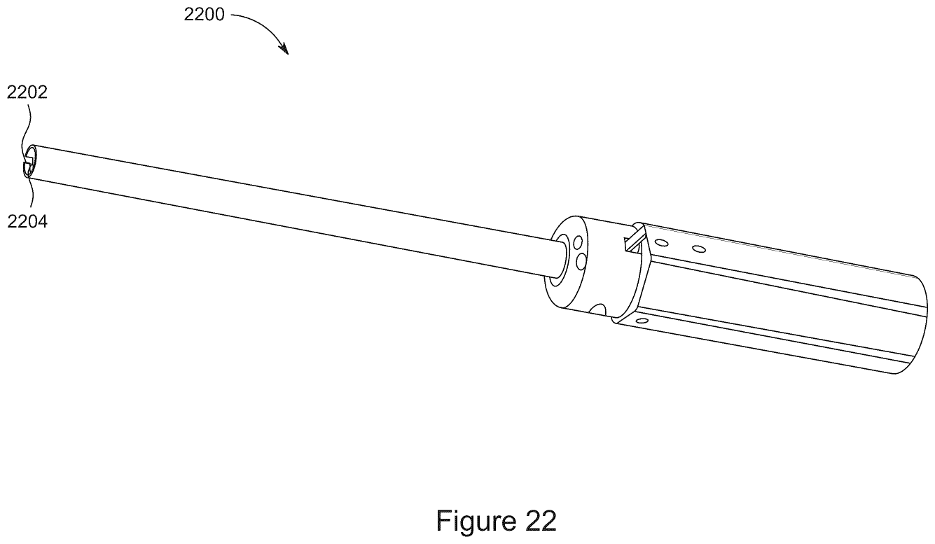

Referring now to FIG. 22, there is shown a side view of an exemplary tendon grabber device 2200, according to some embodiments of the invention. In some embodiments, the tendon can be grabbed using the device 2200 as shown in FIG. 22. The tendon grabber device 2000 includes an axial slot 2202 and a connecting transverse slot 2204 through which a tendon may be inserted when it is desired to retain or move a tendon relative to other anatomical structures or to other devices, according to some embodiments. For example, the device 2000 may be used to hold a tendon and to insert it into a channel of a tendon cutting device such as, for example, channel 325 in device 200 (FIG. 3), according to some embodiments.

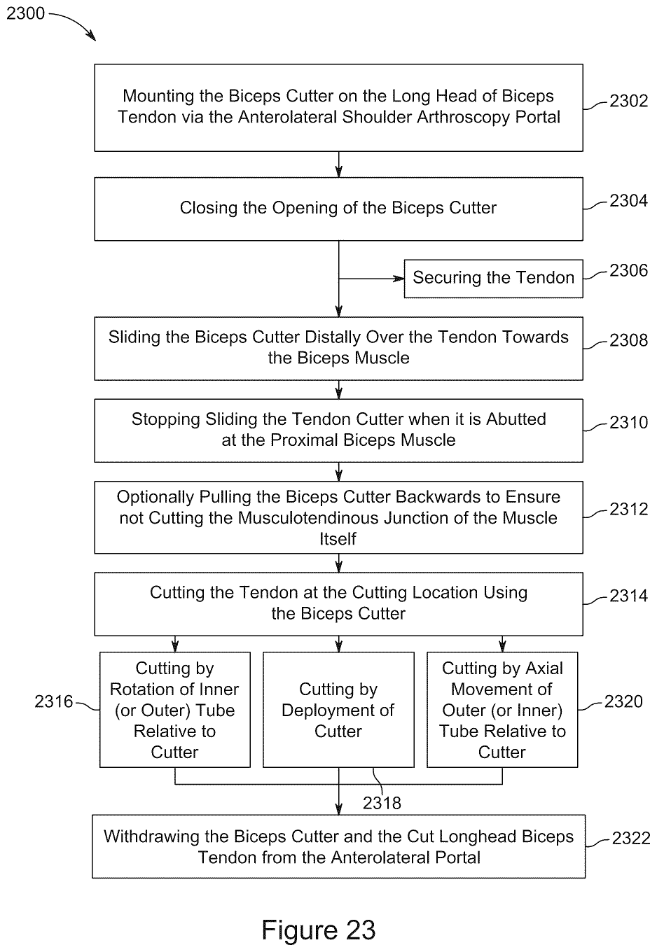

Referring now to FIG. 23, there is shown a flowchart of an exemplary method 2300 for cutting a tendon using a tendon cutting device, according to some embodiments of the invention. In some embodiments, the method 2300 includes, at 2302, mounting the biceps cutter on a tendon such as, for example, the long head of biceps tendon, via an anterolateral shoulder arthroscopy portal; at 2304, optionally closing an opening of the biceps cutter to secure (2306) the tendon within the biceps cutter; at 2308, sliding the biceps cutter distally, relative to a shoulder, over the tendon towards the biceps muscle; at 2310, stopping sliding the tendon cutter when it abuts a proximal portion of the biceps muscle; at 2312, optionally pulling the biceps cutter backwards a distance sufficient to ensure that the musculotendinous junction of the muscle itself is not cut; at 2414, cutting the tendon at a cutting location using the cutter, wherein the cutting is by rotation (2316) of an inner portion of the biceps cutter relative to a cutter (or vice versa), by deployment (2318) of a cutter, or by axial movement (2320) of an outer tube of the biceps cutter relative to a stationary cutter (or axial movement of an inner tube relative to a stationary cutter), to cut the tendon at an oblique angle or at a perpendicular angle to the tendon longitudinal axis; and, at 2322, withdrawing the biceps cutter and a cut long head of biceps tendon from the anterolateral portal.

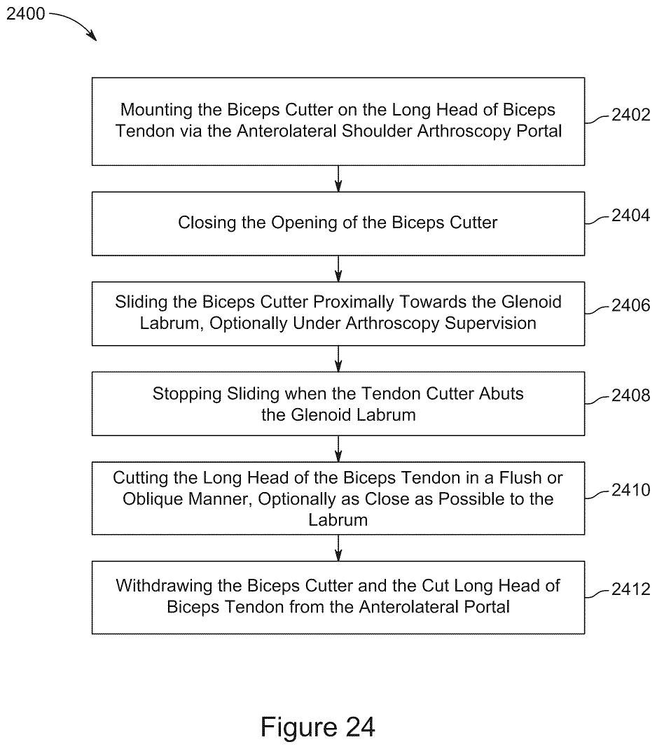

Referring now to FIG. 24, there is shown a flowchart of an exemplary method 2400 for cutting a tendon using a tendon cutting device, according to some embodiments of the invention. In some embodiments, the method 2400, which may be utilized for cutting, for example, a long head of biceps tendon, includes, at 2402, mounting a biceps cutter over a long head of biceps tendon via an anterolateral shoulder arthroscopy portal; at 2404, optionally closing an opening of the biceps cutter; at 2406, sliding the biceps cutter proximally towards a glenoid labrum, optionally under arthroscopic supervision; at 2408, stopping sliding when the biceps cutter abuts the glenoid labrum; at 2410, cutting the long head of biceps tendon in a flush or oblique manner, the cut optionally being done as close as possible to the glenoid labrum; and, at 2412, withdrawing the biceps cutter and a cut long head of biceps tendon via the anterolateral portal.

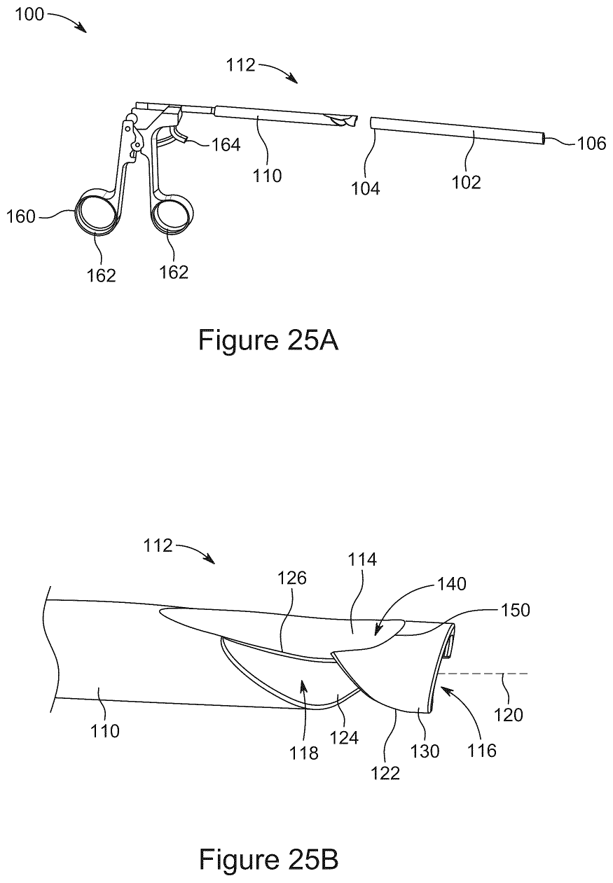

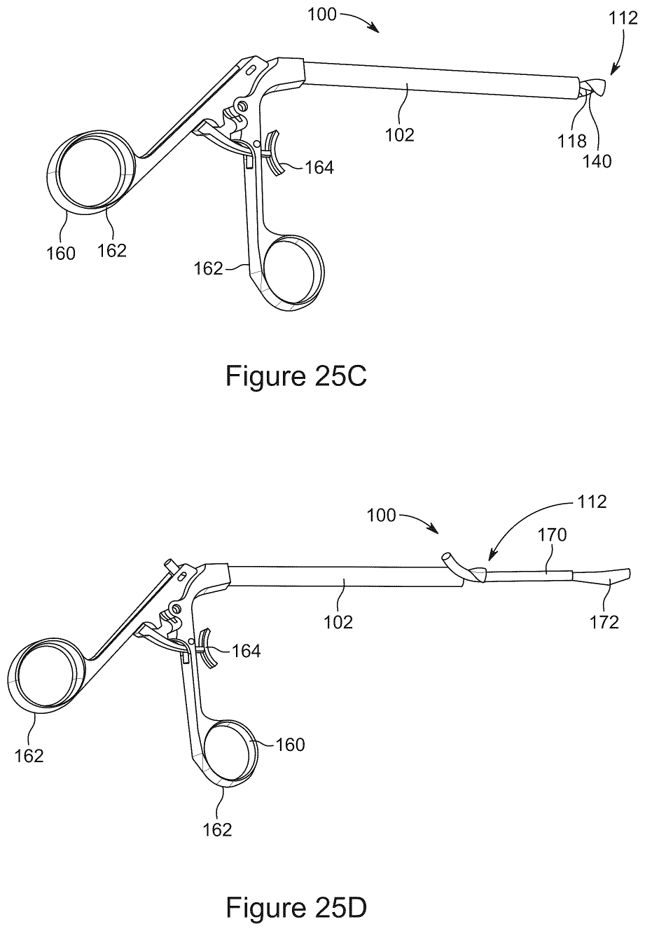

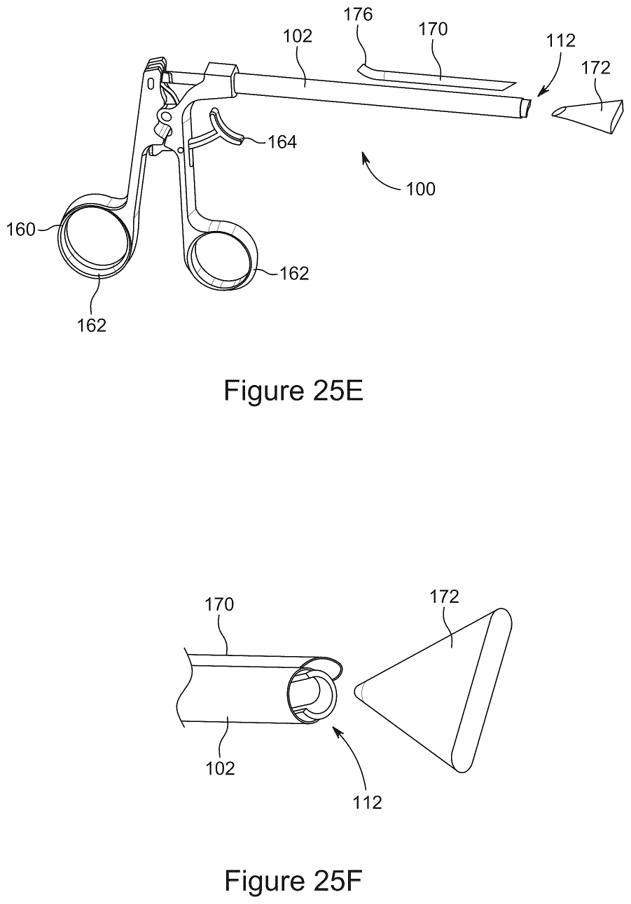

FIG. 25A is an exploded view of an exemplary tendon cutting device 100, according to some embodiments of the invention. According to some embodiments, device 100 includes an outer shaft 102 having a proximal end 104 and a distal end 106, and a stationary inner shaft 110 having a distal end 112, a portion of which has been enlarged at FIG. 25B to show details thereof. Inner shaft distal end 112 is formed with a first inclined or curved surface 114 that is disposed generally at a first angle relative to an inner shaft longitudinal axis 120, according to some embodiments. A flange 130 may be formed along a portion of the inner shaft distal end 112, the flange 130 encompassing less than 360 degrees of the inner shaft distal end 112, according to some embodiments. Flange 130 may define a distal opening 116 on the inner shaft 110, the first inclined surface 114 and flange 130 together forming a passageway 140 for sliding of device 100 along a tendon (not shown), according to some embodiments, as will be discussed further herein. A lateral opening 118 leading to the first inclined surface 114 may be formed in the inner shaft 110, adjacent a first edge 122 of flange 130, and a second inclined or curved surface 124 may be defined on the inner shaft 110 adjacent the first inclined surface 114, according to some embodiments. Between first inclined surface 114 and second inclined surface 124 there may be formed a slightly protruding edge 126, according to some embodiments, and the function of such an edge will be described later herein. It may be noted that second inclined surface 124 may be disposed generally at a second angle relative to longitudinal axis 120, the second angle (between the second inclined surface 124 an the longitudinal axis 120) being larger than the first angle (between first inclined surface 114 and the shaft longitudinal axis 120), according to some embodiments. A cutting edge 150 may be provided on the flange 130, at a location radially outward relative to the first inclined surface 114, according to some embodiments.

With additional reference to FIGS. 25C-D, cutting device 100 is shown in an initial operative orientation, according to some embodiments. A tendon 170 may be mounted on the cutting device by inserting the tendon into passageway 140 via opening 118 (seen most clearly in FIG. 25B).

Device 100 may include a scissors-like actuator 160 having a pair of finger grips 162, according to some embodiments. When finger grips 162 are moved toward each other (to their positions in FIG. 25E), this causes inner shaft 110 to move axially relative to outer shaft 102, in a proximal direction. While not shown in this embodiment, alternatively, a similar tendon cutting device may be configured such that an outer shaft is movable axially, in a distal direction, relative to an inner shaft.

A safety catch 164 may be optionally be provided on device 100, to prevent inadvertent or premature actuation of the device 100. In some embodiments, safety catch 164, which may optionally include a ratchet mechanism, may prevent finger grips 162 from being moved toward each other, thereby preventing inadvertent or premature actuation of the device, until after safety catch 164 has been depressed. The structures and functions of an actuator 160 having finger grips 162 and a safety catch 164 are known to persons skilled in the art, and will not be discussed further herein. It is also to be understood that the particular actuating mechanism and safety mechanism shown and described herein is exemplary only, and that any other suitable actuating mechanism may be utilized, according to some embodiments.

In the initial operative orientation, as shown in FIG. 25D (and with reference to FIG. 25B), a tendon 170 such as, for example, a long head of biceps tendon adjacent a biceps 172, is to be mounted onto device 100, by inserting it via lateral opening 118 and distal opening 116, and alongside second inclined surface 124, such that it is positioned in passageway 140, alongside first inclined surface 114 and extending out through distal opening 116 (see FIG. 25B), according to some embodiments. It may be noted that, while a first end 174 of tendon 170, is shown attached to biceps 172, a second end 176 of tendon 170 may be attached to a shoulder (not shown), for example, at the glenoid labrum or at another anatomical structure of the shoulder. However, in the drawings of this and other embodiments of the tendon cutting device, the shoulder and attachment of second end 176 of tendon 170 thereto have been omitted for the sake of clarity.

Once tendon has been mounted on device 100, the device may be moved along the tendon by sliding the device with the tendon moving through passageway 140, until the device 100 abuts biceps 172, according to some embodiments, and as discussed hereinabove, for example, with regard to the embodiment of FIGS. 3-9. Once so positioned, the device 100 may be actuated by moving finger grips 162 toward each other, from their relative positioned in FIG. 25D (showing device 100 before it has been moved to abut the biceps 172) to their relative positions in FIG. 25E, thereby causing inner shaft 110 to move proximally relative to outer shaft 102, according to some embodiments.

In embodiments including safety catch 164, movement of finger grips 162 toward each other may be limited, and tendon 170 may be retained between outer shaft 102 and flange 130 (FIG. 25B). This may prevent movement of tendon 170 relative to device 100, in some embodiments. Pressure may be applied to safety catch 164 to release it, after which further movement of finger grips toward each other will be enabled.

When finger grips 162 are moved toward each other, for example, from their relative positions in FIG. 25D to their relative positions in FIG. 25E, this will cause the inner shaft 110 to move proximally relative to the outer shaft 102, such that the outer shaft distal end 106 may contact the tendon 170, and may force the tendon against cutting edge 150, which cuts through the tendon, according to some embodiments. The tendon 170 may thus be separated or severed from biceps 172, as shown in FIGS. 25E-F.

It may be noted that, according to some embodiments, device 100 does not require a securing latch or other deployable component for securing tendon 170 within passageway 140. Optionally, tendon 170 may be secured in passageway by the structure of the inner shaft 110, including protruding edge 126, and the flange 130, according to some embodiments. Such securing of the tendon 170 thereby also secures a cutting location on the tendon 170 within the device 100. In some embodiments, device 100 may include an alternative structure adapted for securing the tendon 170 and/or cutting location on the tendon within the device 100.

It will be appreciated by person skilled in the art that, optionally, any of the other embodiments described and shown herein may similarly be structured so as to secure a tendon therewithin such as, for example, a particular inclined or curved surface of a channel, as discussed herein.

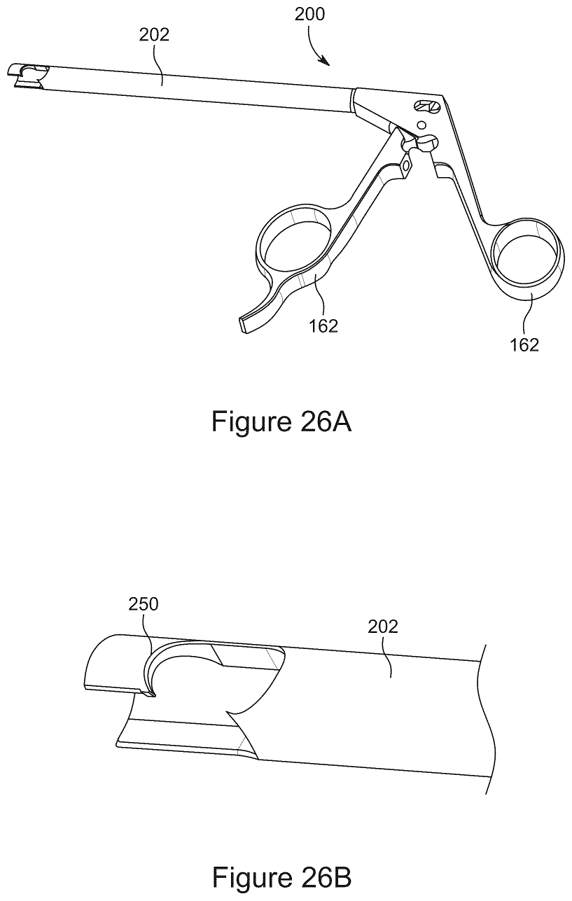

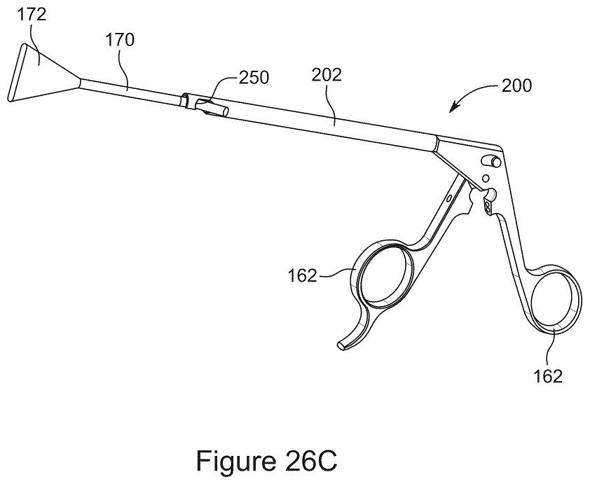

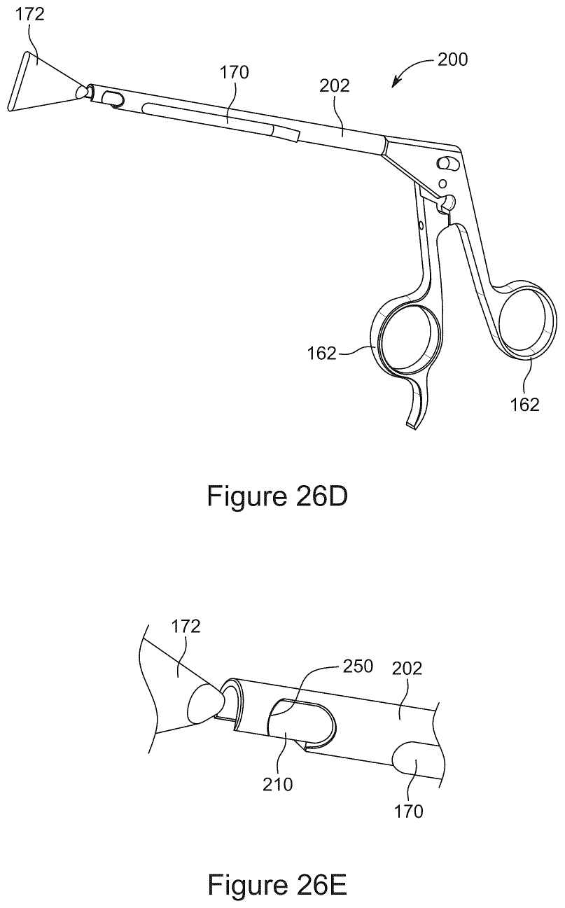

FIG. 26A is a perspective view of an exemplary tendon cutting device 200, according to some embodiments of the invention, and FIG. 26B is an enlargement of a portion of the tendon cutting device 200 shown in FIG. 26A, according to some embodiments of the invention. Device 200 is similar to device 100 (FIGS. 25A-B) in that it includes an outer shaft and an inner shaft. However, in device 100 outer shaft 102 is stationary, and inner shaft 110 is provided with a cutting edge 150 and is axially movable relative to the stationary outer shaft 102. In contrast, in device 200 an outer shaft 202 is stationary and is provided with a cutting edge 250, and an inner shaft 210 is axially movable relative to the outer shaft 202. Additionally, in the embodiment of FIGS. 26A-E, the safety catch (164 in FIG. 25C) has been omitted for simplicity. Optionally, the device of FIGS. 26A-E may include a safety catch similar to that of FIG. 25A.

It will be appreciated by persons skilled in the art that various components of device 200 are similar in structure and function to those of device 100 (FIG. 25A), and they will not be described again with regard to FIGS. 26A-E.

FIG. 26C is a side view of the tendon cutting device 200, shown in an initial operative orientation, according to some embodiments. A tendon 170 may be mounted on the cutting device 200, for example, as discussed herein with regard to FIG. 25D, i.e., by inserting the tendon into a passageway of the device via an opening in the device distal end.

FIG. 26D is a side view of the tendon cutting device 200, shown after cutting the tendon 170, according to some embodiments of the invention, and FIG. 26E shows an enlarged portion of the device 200, according to some embodiments. In FIG. 26D, it can be seen that finger grips 162 have been moved closer together, as compared with their positions in FIG. 26C (at the initial operative orientation). Inner shaft 210, portions of which may be seen more clearly in FIG. 26E, has been moved distally relative to outer shaft, such that inner shaft 210 has forced tendon 170 against cutting edge 250 on outer shaft 202, thereby causing tendon 170 to be separated from biceps 172. While not shown in this embodiment, alternatively, a similar tendon cutting device may be configured such that an outer shaft is movable axially, in a proximal direction, relative to an inner shaft.

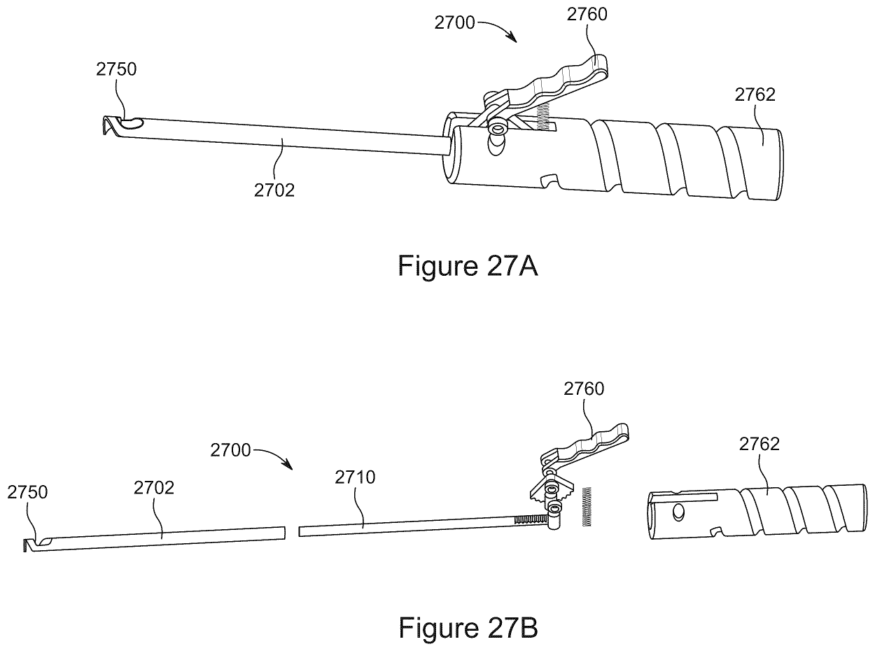

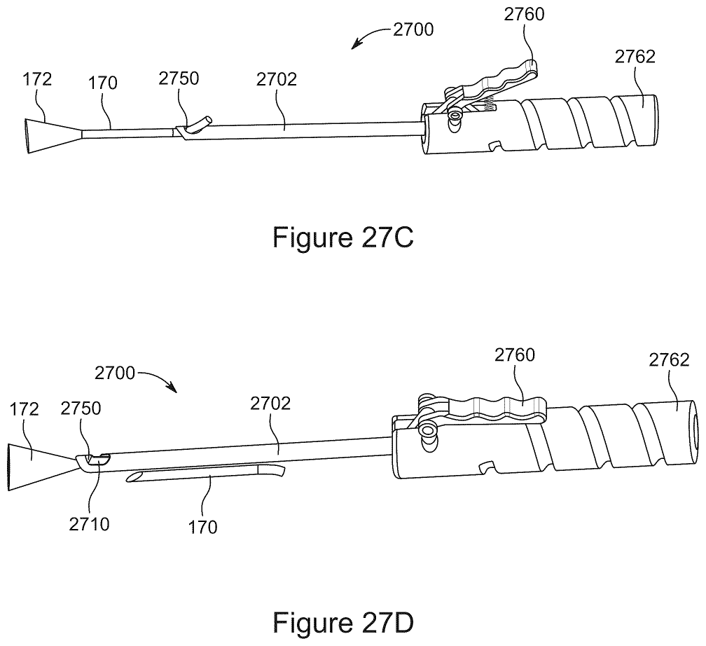

FIG. 27A is a side view of an exemplary tendon cutting device 2700, according to some embodiments of the invention, and FIG. 27B is an exploded view of the tendon cutting device 2700 of FIG. 27A, according to some embodiments of the invention. Device 2700 includes a handle 2762 having an actuation lever 2760, an outer shaft 2702 that is stationary and is provided with a cutting edge 2750, and an inner shaft 2710 that is axially movable relative to the outer shaft 2702.

According to some embodiments, device 2700 may employ a mechanism whereby rotational motion of one or more components of the device is transferred to linear motion of one or more other components of the device, according to some embodiments. For example, device 2700 may include mechanical components for imparting linear motion to inner shaft 2710, as described herein. In some embodiments, the mechanical components may include, for example a toothed element attached to the actuation lever 2760 and the inner shaft 2710 may be provided with corresponding recesses, such that depression of the actuation lever 2760 causes axial movement of the inner shaft 2710. This type of mechanism is known in the art, and will not be described further herein. Additionally, any mechanism for moving inner shaft 2710 axially relative to outer shaft 2702 may be utilized, according to some embodiments.

It will be appreciated by persons skilled in the art that the described mechanism have a toothed element and an element with recesses is merely one example of a mechanism for imparting linear motion to inner shaft 2710. Alternatively any other mechanism for imparting linear motion to inner shaft 2710 in device 2700 may be employed. Similarly, any of the other embodiments described and shown herein may employ such a mechanism for a performing a similar movement of components of the device.

FIG. 27C is a side view of the tendon cutting device of FIG. 27A, according to some embodiments of the invention. Device 2700 is shown in an initial operative orientation, wherein actuation lever 2760 is oriented in a first orientation relative to handle 2762 which, in the embodiment shown, is at an angle relative to the handle. A tendon 170 may be mounted on the cutting device 2700, for example, as discussed herein with regard to FIG. 25C-D, i.e., by inserting the tendon into a passageway of the device via an opening in the device distal end. Components of device 2700 such as, for example, outer shaft 2702, are similar in structure and function to those of device 200 (FIG. 26C), and will not be described further herein. Similarly, some of the actions of mounting of tendon 170 on device 2000 and movement of device 2000 along the tendon toward biceps 172 are similar to those described herein regarding device 200 (FIG. 26C), and will not be described again herein.

Once tendon 170 has been mounted on device 2000, lever 2760 may be moved to a second orientation relative handle 2762 which, in in the embodiment shown in FIG. 27D, is substantially parallel to the handle. According to some embodiments, this movement of lever 2760, from the first orientation (FIG. 27C) to the second orientation (FIG. 27D) may cause inner shaft 2710 to move axially, toward cutting edge 2750. This forces tendon 170 against cutting edge 2750 which cuts through tendon 170, thereby separating it from biceps 172.

With regard to the embodiment of any of FIGS. 27A-D, according to some embodiments, instead of the tendon cutting device having a stationary outer portion including a cutting edge and an axially movable inner portion adapted to force the tendon against the cutting edge, the device may include a stationary inner portion and an axially movable outer portion including a cutting edge adapted to cut the tendon against the stationary portion.

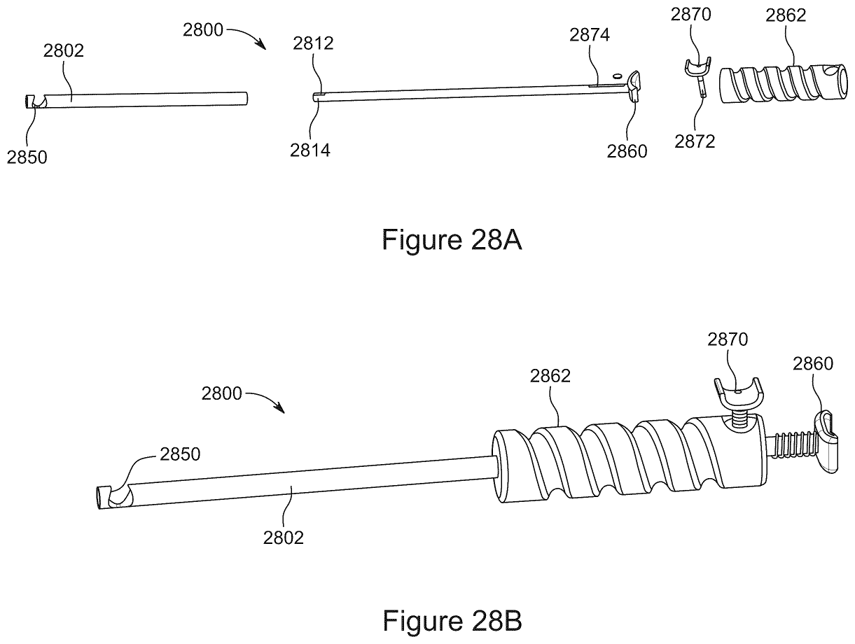

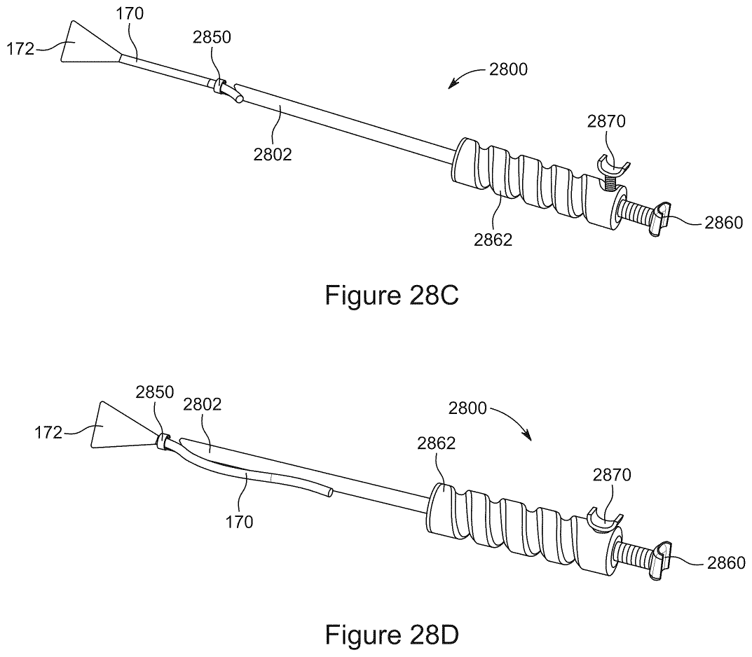

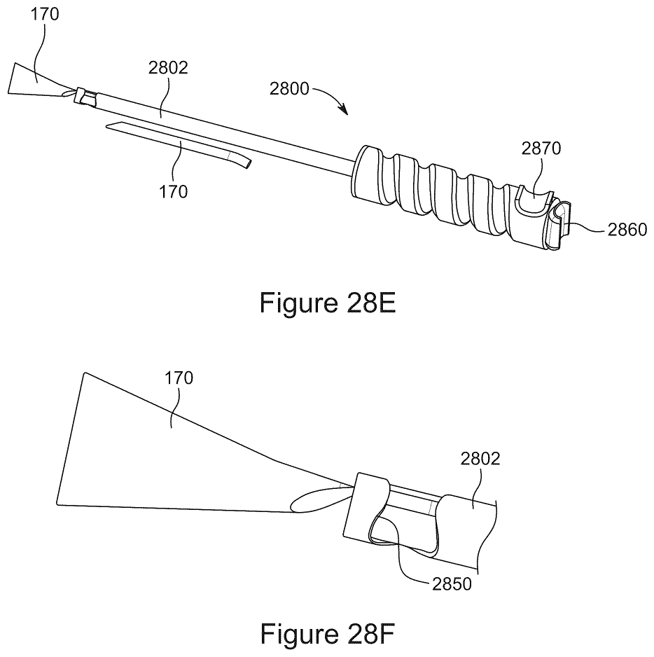

FIG. 28A is an exploded view of an exemplary tendon cutting device 2800 having an actuation button 2860, according to some embodiments of the invention. Device 2800, shown in side view in FIG. 28B, includes a handle 2862 having an actuation switch or button 2860, an outer shaft 2802 that is stationary and is provided with a cutting edge 2850, an inner shaft 2810 that is axially movable relative to the outer shaft 2802, and an optional safety catch 2870, according to some embodiments. Inner shaft 2810 may be provided with a slot 2812 at a distal end 2814 of inner shaft. Slot 2812 will be discussed further with regard to FIG. 28F.

FIG. 28C is a side view of the tendon cutting device 2800 of FIG. 28B, according to some embodiments of the invention. Device 2800 is shown in an initial operative orientation, wherein actuation button 2860 is positioned at a distance from handle 2862. A tendon 170 may be mounted on the cutting device 2800, for example, as discussed herein with regard to FIG. 25C-D, i.e., by inserting the tendon into a passageway of the device via an opening in the device distal end.

Components of device 2800 such as, for example, outer shaft 2802, are similar in structure and function to those of device 200 (FIG. 26C), and will not be described further herein. Similarly, some of the actions of mounting of tendon 170 on device 2800 and movement of device 2800 along the tendon toward biceps 172 are similar to those described herein regarding device 200 (FIG. 26C), and will not be described again herein.

Device 2800 includes a safety feature whereby actuation button 2860 is prevented from being inadvertently or prematurely depressed, thereby preventing inadvertent or premature actuation of the device 2800, according to some embodiments. In particular, device 2800 may include a mechanism whereby there is a geometric interlock between components of the device such as, for example, between a safety catch 2870 and another component of device 2800 such as, for example, actuation button 2860. For example, in some embodiments, a portion 2872 of safety catch 2870 may extend a predetermined distance, optionally through the handle 2862, into a recess 2874 in the actuation button 2860. In some embodiments, when in a locked configuration, the safety catch 2870 may be prevented from moving further into or through the actuation button 2860. After release of the geometric interlock, safety catch 2870 may be allowed to move further into the handle 2862 and/or recess, thereby allowing depression of actuation button 2860 and actuation of device 2800.

It will be appreciated by persons skilled in the art that the described safety catch 2870 is merely one example of a safety mechanism employing a geometric interlock between components. Alternatively any other mechanism for preventing inadvertent or premature actuation of device 2800 may be employed. Similarly, any of the other embodiments described and shown herein may employ such a safety mechanism for a similar purpose.

When a user is ready to cut the tendon 170 which has been mounted on device 2800, after the device has been moved along the tendon such that inner shaft 2810 abuts a biceps 172, safety catch 2870 may be released by pushing it toward handle 2862. Once safety catch 2870 has been released, as shown in FIG. 28D, actuation button 2860 may be depressed. When button 2860 is depressed toward handle 2862, as shown in FIG. 28E, inner shaft 2810 is moved axially, toward cutting edge 2850. This forces tendon 170 against cutting edge 2850 which cuts through tendon 170, thereby separating it from biceps 172, as shown in FIGS. 28E-F.

As noted herein inner shaft 2810 is provided with a slot 2812 at a distal end 2814 of inner shaft, according to some embodiments. It has been discovered that providing inner shaft with a slot enables improved cutting action of the device 2800.

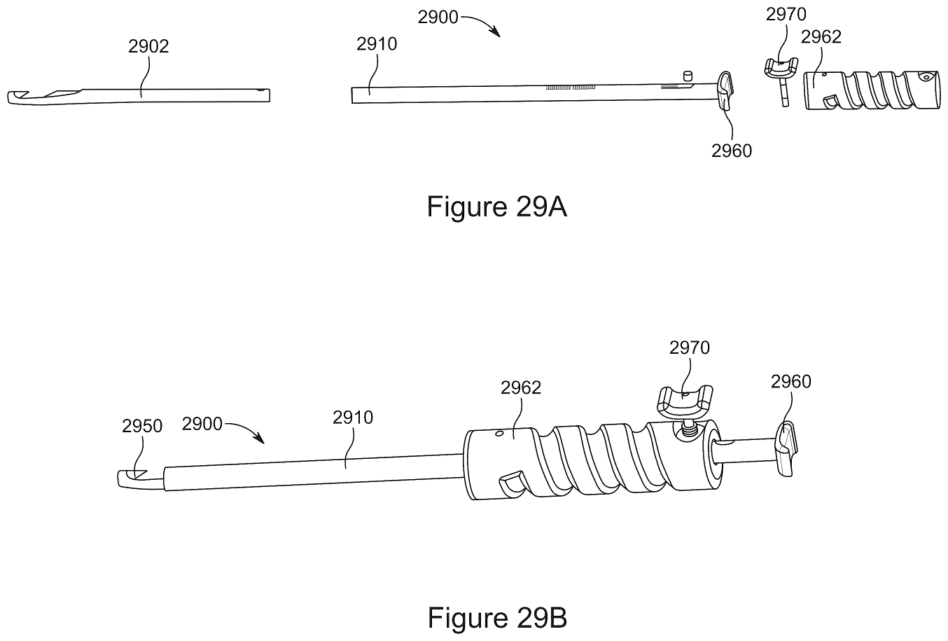

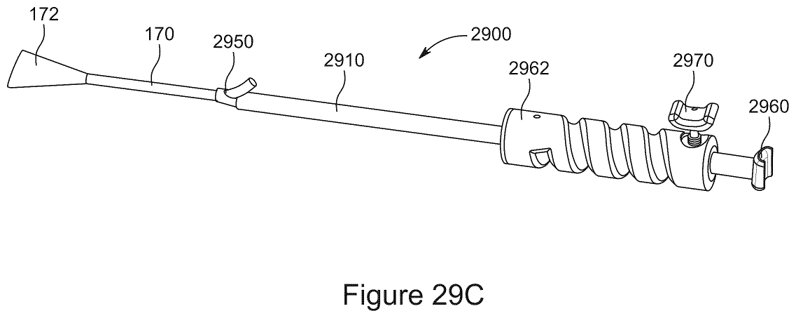

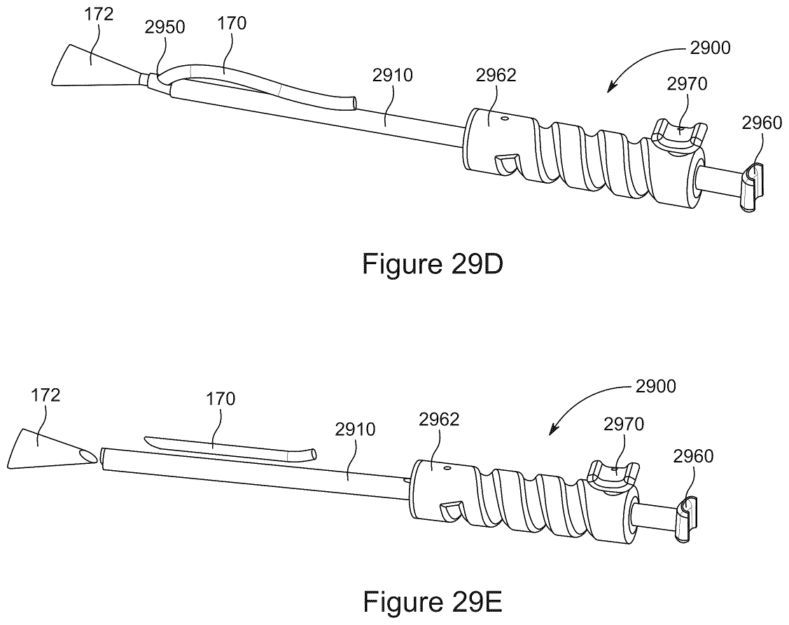

FIG. 29A is an exploded view of an exemplary tendon cutting device 2900 having an actuation button 2960, according to some embodiments of the invention. Device 2900, shown in side view in FIG. 29B, includes a handle 2962 having an actuation switch or button 2960, an inner shaft 2902 that is stationary and is provided with a cutting edge 2950, an outer shaft 2910 that is axially movable relative to the inner shaft 2902, and an optional safety catch 2970, according to some embodiments.

FIG. 29C is a side view of the tendon cutting device 2900 of FIG. 29B, according to some embodiments of the invention. Device 2900 is shown in an initial operative orientation, wherein actuation button 2960 is positioned at a distance from handle 2962. A tendon 170 may be mounted on the cutting device 2900, for example, as discussed herein with regard to FIG. 25C-D, i.e., by inserting the tendon into a passageway of the device via an opening in the device distal end.

Components of device 2900 such as, for example, inner shaft 2902, are similar in structure and function to those of device 100 (FIG. 25C), and will not be described further herein. Similarly, some of the actions of mounting of tendon 170 on device 2900 and movement of device 2900 along the tendon toward biceps 172 are similar to those described herein regarding device 100 (FIG. 25° C.), and will not be described again herein.

Device 2900 may include a safety feature, according to some embodiments, whereby actuation button 2960 is prevented from being inadvertently or prematurely depressed, thereby preventing inadvertent or premature actuation of the device 2900. In particular, device 2900 may include a mechanism whereby there is a geometric interlock between components of the device such as, for example, between a safety catch 2970 and another component of device 2900 such as, for example, actuation button 2960. Components of device 2900 including safety catch 2970 and outer shaft 2910 may be similar in structure and function to that of corresponding components in device 2800 (FIG. 28B) and will not be described again herein.

It will be appreciated by persons skilled in the art that the described safety catch 2970 is merely one example of a safety mechanism employing a geometric interlock between components. Alternatively any other mechanism for preventing inadvertent or premature actuation of device 2900 may be employed. Similarly, any of the other embodiments described and shown herein may employ such a safety mechanism for a similar purpose.

When a user is ready to cut the tendon 170 which has been mounted on device 2900, after the device has been moved along the tendon such that inner shaft 2902 abuts a biceps 172, safety catch 2970 may be released by pushing it toward handle 2962, according to some embodiments. Once safety catch 2970 has been released, as shown in FIG. 29D, actuation button 2960 may be depressed. When button 2960 is depressed toward handle 2962, as shown in FIG. 29E, outer shaft 2910 is moved axially, toward cutting edge 2950. This forces tendon 170 against cutting edge 2950 which cuts through tendon 170, thereby separating it from biceps 172.

With regard to the embodiment of any of FIGS. 25A-29F, according to some embodiments, instead of the tendon cutting device having a stationary portion including a cutting edge and an axially movable portion adapted to force the tendon against the cutting edge, the device may include a stationary portion and an axially movable portion including a cutting edge adapted to cut the tendon against the stationary portion.

It will be appreciate by persons skilled in the art that many of the features described herein may be provided on additional embodiments of the invention. For example, an embodiment having a rotatable cutter as described here may, alternatively be provide with a deployable cutter as described herein; or may, alternatively, be provided with a stationary cutter and an axially movable portion that forces a tendon against the stationary cutter, as described herein. For example, embodiments as discussed herein having an actuation lever, as discussed herein, may alternatively be provided with an actuation button, as discussed herein, or alternatively with an actuator having finger grips, as discussed herein. Additionally, any of the devices discussed herein may be provided with laterally-positioned grips, such as, for example, located on a handle of the device, for facilitating gripping of the device. It is expected that during the life of a patent maturing from this application many relevant tendon cutting devices will be developed; the scope of the term tendon cutting device is intended to include all such new technologies a priori. It is understood that additional devices and methods combining elements of the above embodiments may be employed without straying from the spirit of the invention.

As used herein with reference to quantity or value, the term “about” means “within 20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for cutting an end portion of a long head of biceps (LHB) tendon from a biceps, comprising:
a. mounting a tendon cutting device having a cutter to the LHB tendon;
b. moving said tendon cutting device in a distal direction relative to a shoulder along the LHB tendon towards the biceps;
c. abutting said tendon cutting device to the biceps, thereby aligning a cutter location associated with a distal end of said tendon cutting device with a cutting location on the LHB tendon adjacent the biceps; and
d. cutting the LHB tendon at said cutting location using said cutter.

2. The method according to claim 1, further including securing the LHB tendon within said tendon cutting device after said mounting.

3. (canceled)

4. The method according to claim 2, wherein said securing includes at least one of:
securing the LHB tendon within a securing channel of said tendon cutting device; and
closing an opening of the tendon cutting device to secure the cutting location within said biceps cutter.

5. The method according to claim 1, wherein said cutting is performed by relative rotation of said cutter.

6. The method according to claim 5, wherein said tendon cutting device includes inner and outer portions rotatable relative to each other, said inner portion located radially inward relative to said outer portion, and wherein said cutter is located on one of:
said inner portion; and
said outer portion.

7. (canceled)

8. The method according to claim 1, wherein said cutter is stationary within said tendon cutting device and said cutting is performed by rotation of a rotatable portion of the tendon cutting device relative to said cutter.

9. The method according to claim 8, wherein said tendon cutting device includes inner and outer portions rotatable relative to each other, said inner portion located radially inward relative to said outer portion, and wherein said cutter is located on one of:
said inner portion; and
said outer portion.

10. (canceled)

11. The method according to claim 1, wherein said cutting is performed by moving said cutter at a predetermined angle relative to the LHB tendon.

12-13. (canceled)

14. The method according to claim 1, wherein said cutter is defined on a stationary portion of said tendon cutting device and wherein said cutting is performed by moving a movable portion of said tendon cutting device axially toward said cutter, said movable portion forcing the LHB tendon against said cutter.

15. The method according to claim 14, wherein said stationary portion is positioned one of:
radially outward relative to said movable portion; and
radially inward relative to said movable portion.

16. (canceled)

17. The method according to claim 2, wherein said securing is performed after or simultaneously with pushing a first release handle associated with said tendon cutting device.

18. The method according to claim 5, wherein said rotation is performed after or simultaneously with pushing a second release handle associated with said tendon cutting device.

19. The method according to claim 11, wherein said moving is performed by pushing a handle associated with said tendon cutting device.

20. The method according to claim 1, wherein said cutter is a mechanically deployable knife.

21. The method according to claim 1, wherein said mounting includes mounting the tendon cutting device on the LHB_tendon via an anterolateral shoulder arthroscopy portal;
wherein said moving includes sliding the tendon cutting device distally relative to the shoulder over the LHB tendon;
wherein said sliding is stopped when said biceps cutter abuts the biceps;
wherein said cutting is by relative rotation, at an oblique angle or at a perpendicular angle relative to a LHB tendon longitudinal axis; and
wherein said method includes withdrawing the tendon cutting device and a cut LHB tendon from the anterolateral shoulder arthroscopy portal.

22. The method according to claim 21, further including, after said stopping, moving the tendon cutting device proximally relative the shoulder a sufficient distance to ensure that a musculotendinous junction of the biceps muscle is not cut by said tendon cutting device.

23. (canceled)

24. A device for cutting an end portion of a long head of biceps (LHB) tendon from a biceps, comprising:
a. an elongate body mountable on the LHB tendon, said elongate body including a tip and a cutter at a distal end of said body, said tip shaped as a channel to receive a portion of the LHB tendon and adapted to move along the LHB tendon and to cease the motion on the LHB tendon when a distal end of said tip abuts a thickened portion of the biceps; and
b. a cutter location associated with said body and located at a predetermined distance from said distal end of said tip, said cutter configured to cut the LHB tendon at said cutter location.

25. (canceled)

26. The device according to claim 24, wherein said cutter location is at a distance of from about 1 mm to about 5 mm from said distal end of said tip.

27-29. (canceled)

30. The device according to claim 24, wherein said device includes a stationary portion and a movable portion, wherein said cutter is defined on said stationary portion, and wherein said movable portion is configured to be moved toward said cutter and is configured to force the tendon against said cutter.

31. The device according to claim 30, wherein said stationary portion is positioned one of:
radially outward relative to said movable portion; and
radially inward relative to said movable portion.

32. (canceled)

33. The device according to claim 30, wherein said movable portion is configured to one of move axially and rotate.

34. The device according to claim 24, wherein said device includes a stationary portion and a movable portion, wherein said cutter is defined on said movable portion, and wherein said movable portion is configured to be moved toward said stationary portion and is configured to force the LHB tendon against said stationary portion.

35. The device according to claim 34, wherein said stationary portion is positioned one of:
radially outward relative to said movable portion; and
radially inward relative to said movable portion.

36. (canceled)

37. The device according to claim 34, wherein said movable portion is configured to one of move axially and rotate.

38. The device according to claim 24, further including a securing bar for securing the LHB tendon within said channel.

39-41. (canceled)

42. The device according to claim 24, wherein said cutter is at least one of:
deployable; and
rotatable.

43-54. (canceled)

55. The method according to claim 1, wherein said abutting includes abutting the tendon cutting device to one of:
a thickened portion of the biceps;
a widened portion of the biceps;
a proximal portion of the biceps; and
a glenoid labrum.
